(19) United States
(12) Patent Application Publication
(10) Pub. No.: US 2025/0229080 A1
(43) Pub. Date: Jul. 17, 2025

(54) WEARABLE ELECTRONIC FOR DIGITAL HEALTHCARE

(71) Applicant: John James Daniels, Madison, CT (US)

(72) Inventor: John James Daniels, Madison, CT (US)

(21) Appl. No.: 18/854,258

(22) PCT Filed: Apr. 4, 2023
(86) PCT No.: PCT/US23/65316

§ 371 Date: Oct. 4, 2024

(51) Int. Cl. A61N 1/04; A61N 1/36; G16H 10/60; G16H 20/10; G16H 20/40; G16H 40/60; G16H 50/30

(52) U.S. Cl. CPC A61N 1/0484; A61N 1/0452; A61N 1/36031; G16H 10/60; G16H 20/10; G16H 20/40; G16H 40/60; G16H 50/30

(57) ABSTRACT

A wearable electronic medical device that stores initial baseline biometric data in memory and determines patient-specific thresholds for monitored biometric parameters. Biometric sensors detect changes in these parameters in response to therapeutic treatment or disease progression, and exceeded thresholds activate actions such as patient notifications, changes in treatment, and transmission of data. Trusted receivers receive suggested diagnoses and treatment options determined by an AI-powered web crawler. The treatment includes electrocuetical and pharmaceutical treatment, and the physiological change includes an indication of a change in the cardiovascular condition. The device determines patient-specific thresholds by applying statistical weighting to monitored biometric parameters and determining an acceptable data set.

### CROSS-REFERENCE TO RELATED APPLICATIONS

This application claims priority to and the benefit of priority of U.S. Provisional Patent application Ser. No. 63/327,921, entitled Wearable Electronic For Monitoring a Change in a Physical Condition of a Patient, filed 6 Apr. 2022, the which prior application is incorporated herein in its entirety.

### BACKGROUND

The exemplary and non-limiting embodiments of this invention relate generally to digital therapeutic systems, methods, devices and computer programs and, more specifically, relate to digital therapeutic wearable electronic garments for detecting and reporting a biometric and automatically adjusting an applied therapy in response thereto.

The present invention also pertains to a device architecture, specific-use applications, and computer algorithms used with wearable electronics in the form of clothing and other wearable garments with the capability to detect biometric parameters for the treatment and monitoring of physiological conditions in humans and animals, such as for the prevention, treatment and/or prophylaxis of venous thromboembolism or deep vein thrombosis.

This section is intended to provide a background or context to the exemplary embodiments of the invention as recited in the claims. The description herein may include concepts that could be pursued but are not necessarily ones that have been previously conceived, implemented or described. Therefore, unless otherwise indicated herein, what is described in this section is not prior art to the description and claims in this application and is not admitted to be prior art by inclusion in this section.

An event-related potential (ERP) is the measured brain response that is the direct result of a specific sensory, cognitive, or motor event. Electrical Muscle Stimulation (EMS), or Transcutaneous Electrical Nerve Stimulation (TENS), can be applied as a form of physical therapy and/or electro-analgesia. Unless specified, the acronyms TENS and EMS may be used interchangeably herein as denoting electrical signals applied through the skin to the underlying muscles and/or nerves of the user.

A typical TENS unit includes a battery powered electrical signal generator with long wires that connect with a set of gel electrodes. Self-adhesive electrodes use a hydrogel to make contact between a conductive member and the surface of the user's skin. The gel electrode is typically built in a multi-layer configuration sometimes including multiple layers of hydrogel. The skin interface layer may include an electrically conductive gel for removably contacting the user's skin. The conductive gel is made from co-polymers derived from polymerization, e.g. of acrylic acid and N-vinylpyrrolidone. In a multiple layer hydrogel, a second hydrogel layer connects a substrate conductive member (a low resistive material such carbon impregnated rubber or a wire mesh) with the skin hydrogel layer.

A typical TENS unit is able to generate signals with variable current strengths, pulse rates, and pulse widths. A sometimes preferred waveform is biphasic, to avoid the electrolytic and iontophoretic effects of a unidirectional current. The usual settings for the stimulus parameters used clinically include amplitude (signal intensity), pulse width (duration), and pulse rate (frequency).

When TENS is used analgesically, electrode positioning is an important consideration. The electrodes may be placed on or near the painful area, or at other locations (for example, at cutaneous nerves, trigger points, acupuncture sites). Medical complications arising from use of TENS are rare. However, skin irritation often occurs due, at least in part, to drying out of the electrode gel and to the salt and other ingredients comprising the conductive hydrogel.

The conventional sticky gel electrode is a relatively expensive component that needs to be replaced often. Salts and other materials in the hydrogel can irritate the skin. The removal of the sticky gel electrode is often very discomforting, especially when applied over hair. Also, the sticky gel electrode become dirty and very quickly loses the ability to adhere to the skin.

The wires required to conduct the electrical signal from the TENS unit to the gel electrodes are cumbersome and often get entangled and either disconnect the gel electrode from the TENS unit, or pull the gel electrode off the user's skin. These wires are particularly inconvenient if the user wishes to have mobility while using the TENS treatment.

Accordingly, there is a need for a more convenient TENS system that avoids the drawbacks of the conventional sticky gel electrodes and avoids the need for long, loose wires to conduct the TENS signal from the TENS generator to the electrodes.

In the US, there are 900,000 people affected by deep vein thrombosis (DVT) and/or pulmonary embolism (PE). 1 out of 9 of these DVT/PE patients will die as a result of their condition. Each year, more people die of DVT/PE than die from breast cancer, traffic accidents and HIV combined.

Clotting agents in blood, platelets (thromobcytes) and fibrin, are present to prevent blood loss. However, problems arise when blood clots lodged in blood vessels of the lower legs travel to the lungs. The treatment of deep vein thrombosis (DVT) is intended to prevent the clot from getting bigger and preventing it from breaking loose and causing a pulmonary embolism. Pharmacological treatment options include blood thinners or anticoagulants that decrease the blood's ability to clot.

Clot buster drugs or thrombolytics may be prescribed to break up clots quickly but are generally reserved for severe cases of blood clots. A vena cava filter may be implanted to catch clots that break loose from lodging in the lungs, and compression stockings are typically worn to help prevent swelling associated with deep vein thrombosis, these are often worn on the legs from the feet to about the level of the knees.

To heal an injury to a vein or artery, the body uses platelets (thrombocytes) and fibrin to clot the blood and prevent blood loss. Blood clots also can form within a blood vessel even when there is no injury. Thrombosis occurs when a blood clot formed inside a blood vessel obstructs the flow of blood through the circulatory system. If the clot is anchored in place within the blood vessel it may eventually dissolve without any issue. But, if the clot breaks free and begins to travel around the body, life threatening damage can occur. The dislodged clot, an embolus, can lodge within the circulatory system causing a type of embolism known as a thromboembolism.

DVT most commonly affects leg veins and occurs when a blood clot forms within a deep vein. A venous thromboembolism (VTE) can lodge in the lung causing a debilitating and often fatal pulmonary embolism (PE). A PE occurs when a DVT blood clot dislodges from a blood vessel and becomes lodged in the lungs. A PE that blocks blood flow can be life threatening, damaging the lungs and other organs. The symptoms of PE include shortness of breath, pain with deep breathing, and coughing up blood. Some people experience these symptoms, unaware that they may have started as a deep vein blood clot. About 380 million people, or 5% of the world's population, is affected by DVT and VTE as some point in their lives.

The standard pharmacological treatment for thrombosis is anticoagulation to reduce the ability of platelets and fibrin to interact and cause blood clotting. Rivaroxaban, developed by Bayer and sold under the brand name Xarelto, is the first orally administered medication with a direct Factor Xa inhibitor. Factor Xa is a chemical part of the body's coagulation mechanism. In 2011, the US FDA approved Rivaroxaban for stroke prevention in people with non-valvular atrial fibrillation. In 2012, the FDA approved Xarelto for treatment of deep vein thrombosis and pulmonary embolism.

A review of the patent literature shows the use of an electrical stimulator for the prevention of deep vein thrombosis, ankle edema, and venostasis. U.S. Pat. No. 5,653,331, entitled Method and device for prevention of deep vein thrombosis, issued Jul. 1 1997 to Amiram Katz, shows the use of an anode and cathode electrode pair secured at or near the tibial nerve at the popliteal fossa on both legs of a patient. An electrical signal is applied to stimulate the nerve causing muscle contractions in the calf of the legs to prevent deep vein thrombosis, ankle edema, and venostasis.

PCT patent application PCT/US99/08450, entitled Neuromuscular electrical stimulation for preventing deep vein thrombosis, applied for by Stryker Instruments, International filing date 16 Apr. 1999 shows a neuromuscular electrical stimulation system that instigates muscle twitch to prevent DVT. The duration and duty cycle of the applied electrical pulses are controlled to instigate the muscle twitch without causing tetanic, or full and sustained, muscle contractions.

Venography is the current standard for diagnosing DVT, where a special dye is injected into the bone marrow or veins. The dye has to be injected constantly via a catheter, making it an invasive procedure. Light Reflection Rheography (LRR) is a non-invasive technique that uses LEDs and a sensor to measure DVT with the LEDs and sensors at the skin surface. The intensity of the reflected light quantifies the venous function by measuring changes in microcirculation.

U.S. Pat. No. 5,282,467, entitled non-invasive method for detecting deep venous thrombosis in the human body, issued Feb. 1, 1994 to Piantadosi et al. shows a non-invasive method for detecting deep venous thrombosis, a change in the amount of deoxyhemoglobin can be detected by trapping blood in a vein for a determined time period. Light sources are used to emit two selected wavelengths that penetrate into the deep venous system. The reflectance contribution of the selected wavelengths are used to measure changes in blood flow and amount of deoxyhemoglobin indicative of presence or absence of deep venous thrombosis.

The prior attempts at mitigating a circulatory and/or cardiovascular disease, such as DVT, all fall short of optimizing the cost, patient outcome, and general advantageous collection of biometric information. According there is a need for digital therapeutic systems, methods, devices and computer programs for detecting and reporting a biometric and automatically adjusting an applied therapy in response thereto.

It has been stated that “the organism is an algorithm.” Therefore, subject to Moore's law and exponential growth, the implications are that information technology and digital health will play a surprisingly rapid and increasingly important role in global healthcare—for humans and animals. Accordingly, there is a need for a digital therapeutic device that is capable of the detection and analysis of biometric parameters, and the modification of treatments based on the biometric parameters, to enable digital healthcare options.

Current approaches to mitigating circulatory and cardiovascular diseases like DVT fall short in terms of optimizing cost, patient outcomes, and the collection of biometric information. Traditional TENS units that use sticky gel electrodes and long wires to conduct electrical signals are inconvenient and can cause skin irritation. Invasive diagnostic techniques like venography require the injection of a special dye, while non-invasive methods like light reflection rheography have limitations in terms of accuracy and ease of use.

There is a growing need for digital therapeutic systems that can detect and analyze biometric parameters, modify treatments based on these parameters, and enable digital healthcare options. By leveraging advances in information technology and digital health, the invention aims to provide a more convenient and effective solution for preventing and treating circulatory and cardiovascular diseases. The wearable electronic garments can detect biometric parameters, automatically adjust applied therapies, and collect data for further analysis and treatment optimization.

### BRIEF SUMMARY

The below summary section is intended to be merely exemplary and non-limiting. The foregoing and other problems are overcome, and other advantages are realized, by the use of the exemplary embodiments of this invention.

The present invention relates to digital therapeutic wearable electronic garments for monitoring and treating physiological conditions in humans and animals, particularly for the prevention and treatment of deep vein thrombosis. The invention provides a wearable electronic that detects a change in a physical condition of a patient and compares it with a baseline biometric. The initial baseline biometric data is obtained using a baseline biometric test and stored in memory. A patient-specific threshold is determined for a monitored biometric parameter dependent on the stored baseline biometric data. The monitored biometric parameter is detected using a biometric sensor and is dependent on a physiological change of a patient occurring after the baseline biometric data is obtained. An action is activated depending on the determined exceeded threshold, which may include notifying the patient or trusted receiver, or changing the therapeutic treatment. The invention allows for real-time monitoring and personalized treatment for patients, leading to improved patient outcomes and reduced healthcare costs.

An aspect of the invention comprises a wearable electronic device that monitors a patient's physical condition and compares it with a baseline biometric specific to that patient. The device uses biometric sensors to detect changes in the patient's physiological parameters that occur in response to a therapeutic treatment, change in health condition, or progression of a disease. The device determines patient-specific thresholds for monitored biometric parameters based on the stored baseline data and activates an action, such as a notification to the patient or a change in treatment, if a threshold is exceeded. The device can be used in a variety of medical and non-medical applications, and can be configured in different embodiments such as an elastic wrap with electrodes for applying stimulation electrical signals to the skin of a user.

This aspect of the invention can be used in various medical and non-medical use cases, such as monitoring and managing various physiological conditions and diseases, including but not limited to thrombotic events, cardiovascular conditions, diabetes, chronic pain, and sleep disorders. The wearable electronic device can also be used to monitor and improve athletic performance, physical therapy, and rehabilitation. Additionally, the device can be used for data collection and analysis for research purposes. The invention has a wide range of applications in the healthcare and wellness industries.

### BRIEF DESCRIPTION OF THE SEVERAL VIEWS OF THE DRAWINGS

To easily identify the discussion of any particular element or act, the most significant digit or digits in a reference number refer to the figure number in which that element is introduced.

FIG. 1 is a flow chart illustrating an algorithm for activating an action based on comparison of a baseline biometric versus monitored biometrics.

FIG. 2 shows smartphone graphical user interface screens of monitored biometrics.

FIG. 3 illustrates the location of various biometric detectors/sensors/transmitters/processors/actuators on the lower legs of a patient.

FIG. 4 illustrates an embodiment of an electroceutical combination treatment device for applying an electroceutical signal in combination with detecting a biometric physiological response.

FIG. 5 illustrates an embodiment of an electroceutical treatment device for monitoring physiological changes in response to an administered treatment.

FIG. 6 is a flow chart illustrating an algorithm for applied probabilistic analysis to determine a concerning physiological change.

FIG. 7 is a flow chart illustrating an algorithm for an early warning system with applied probabilistic analysis of multiple biometric parameters.

FIG. 8 is a flow chart illustrating an algorithm for a single parameter early warning system.

FIG. 9 is a flow chart illustrating an algorithm for biometric fusion analysis of multiple biometrics to determine a physiological change.

FIG. 10 is a photo showing an embodiment of a wearable electronic for DVT prevention and remote patient monitoring.

FIG. 11 illustrates the wearable electronic for DVT prevention with biometric sensors for EMG and EKG, temperature and swelling detection.

FIG. 12 is a drawing showing a wrap embodiment with foam fit electrodes for applying an EMS signal and receiving EMG, heart, or other electrical biometric signals from the same electrodes.

FIG. 13 is a drawing showing removable electronics disposed on the wrap embodiment.

FIG. 14 is a drawing illustrating the placement of the wrap embodiment onto the lower leg of a patient.

FIG. 15 is a block diagram showing dual data paths for privacy ensured data acquisition and utilization.

FIG. 16 shows a swelling detector made by printing stretchable conductive ink on a stretch fabric.

FIG. 17 is a side view of the swelling detector.

FIG. 18 is an exploded side view of the swelling detector.

FIG. 19 illustrates the detectable increase in resistance when conductive particulate of the swelling detector are separated as swelling occurs in a lower leg.

FIG. 20 is a top view showing the relative dimensions in inches of an embodiment of electronics with snap connectors for electrically and mechanically mounting on the wearable electronic.

FIG. 21 is a top view of the electronics.

FIG. 22 is a perspective view of the electronics.

FIG. 23 shows a top view of electronics with multiple in-line snap connections

FIG. 24 is a side view showing snaps and flex circuit PCB before a crimping operation.

FIG. 25 is a side view showing snaps and flex circuit PCB after the crimping operation.

FIG. 26 shows multiple biosensors for detecting biometric parameters indicative of a thrombotic condition and/or diseases progression.

FIG. 27 shows the placement of biometric sensors on the lower legs of a patient.

FIG. 28 is a flowchart illustrating an automatic muscle pump treatment algorithm.

FIG. 29 continues the flowchart shown FIG. 28.

FIG. 30 continues the flowchart shown in FIG. 29.

FIG. 31 shows the location of multiple temperature sensors for creating a heat map of a rise in temperature on the lower leg of a patient.

FIG. 32 shows a thermal harvesting construction of the wrap embodiment for DVT prevention with thermally conductive stretch fabric for conducting a temperature increase at one local area of the lower leg to a temperature sensor at another location.

FIG. 33 illustrates a construction of a wearable electronic wrap having an elastic substrate that adheres to itself and not to skin, along with a skin adhesive strip for anchoring the substrate to facilitate wrapping around a body part.

FIG. 34 shows the wearable electronic wrap having two conductive stretch fabric skin contact electrodes.

FIG. 35 shows the wearable electronic wrap having a small, mobile, bluetooth enabled electronics and rechargeable battery package.

FIG. 36 shows the wearable electronic wrap having conductive snaps for electrically communicating the skin contact electrodes with the electronics.

FIG. 37 shows the wearable electronic wrap having an end being anchored by the skin adhesive strip to the forearm of a user.

FIG. 38 shows the anchored wearable electronic wrap being wrapped using one hand of the user around the user's other forearm.

FIG. 39 shows the wearable electronic wrap anchored around the forearm of the user.

FIG. 40 shows the hand of the user in a resting position with an APP enabled smartphone used for controlling the bluetooth enabled electronics.

FIG. 41 shows the hand of the user pivoting at the wrist to stretch the muscles and tendons of the forearm to prevent contracture and atrophy.

FIG. 42 illustrates an embodiment of a Haptic Human/Machine Interface (HHMI™) wearable electronic configured for upper limb disability recovery use-cases.

FIG. 43 illustrates an embodiment of the HHMI wearable electronic in combination with a VR headset with forward looking cameras for simultaneously applying involuntary muscle movement and haptic sensation with synchronized virtual reality audio and video sensory cues.

FIG. 44 illustrates an HHMI pixelated shirt in combination with forward looking cameras and VR/AR/XR head gear.

FIG. 45 illustrates the HHMI pixelated shirt for a haptic vision use-case.

FIG. 46 shows a scene as a visually impaired user wearing the HHMI pixelated shirt approaches a cross walk.

FIG. 47 shows the scene of the cross walk reproduced as haptic sensations on skin of the torso of the visually impaired user.

FIG. 48 shows a smart shirt having a skin-contact information display system comprising individually addressable electrodes for applying electrical signals and/or haptic sensations, such as vibrations using localized and addressable vibrators.

FIG. 49 shows the scene of the cross walk at the farther distance from the visually impaired user walking towards the other side of the street.

FIG. 50 shows the scene of the cross walk as the visually impaired user comes closer to the other side of the street.

FIG. 51 shows the scene of the cross walk as the visually impaired user comes even closer to the other side of the street.

FIG. 52 illustrates how the scene of the cross walk is reproduced on there HHMI pixelated shirt as some scene elements remain stationary and some are in motion.

FIG. 53 illustrates how the scene reproduced on the HHMI pixelated shirt indicates to the visually impaired user where elements of the scene are in motion and others remain stationary.

FIG. 54 illustrates how elements of the scene are proportionally increased in size and haptic signal intensity as the visually impaired user approaches the other side of the street.

FIG. 55 shows locations of skin contact electrodes and position detectors for an embodiment of the wearable electronic garment used for gait disorders.

FIG. 56 shows some of the upper leg muscles that can be used as Balance Control Muscles.

FIG. 57 shows a pair of shorts with skin contact electrodes for applying EMS signals to BCMs of the upper legs.

FIG. 58 is a flowchart for AI/ML adjusted therapy.

### DETAILED DESCRIPTION

Below are provided further descriptions of various non-limiting, exemplary embodiments. The exemplary embodiments of the invention, such as those described immediately below, may be implemented, practiced or utilized in any combination (e.g., any combination that is suitable, practicable and/or feasible) and are not limited only to those combinations described herein and/or included in the appended claims. Throughout this application, an applied electrical signal is denoted as being a TENS, EMS, NMES, or other acronym. The difference among the applied electrical signal may be one of frequency or other signal characteristic and unless specified or otherwise inferable through the descriptive context, the terms and acronyms used for the applied electrical signal may be considered interchangeable with each other. For example, TENS will typically be used when describing an electrical signal applied for pain mitigation however, as used herein it might also be used when describing a signal that invokes an involuntary muscle contraction.

Many configurations, embodiments, methods of manufacture, algorithms, electronic circuits, microprocessor, memory and computer software product combinations, networking strategies, database structures and uses, and other aspects are disclosed herein for a wearable electronic digital therapeutic device and system that has a number of medical and non-medical uses. A non-limiting exemplary embodiment is shown in the drawing figures, for example, as an apparatus that comprises an elastic support or wrap embodiment with a pair of electrodes supportable by the elastic support. The electrodes apply stimulation electrical signals to the skin of a user. At least one urging member is supportable by the elastic support adjacent to the electrodes for urging the electrodes towards the skin of the user to ensure adequate surface area contact between the electrode and the skin surface.

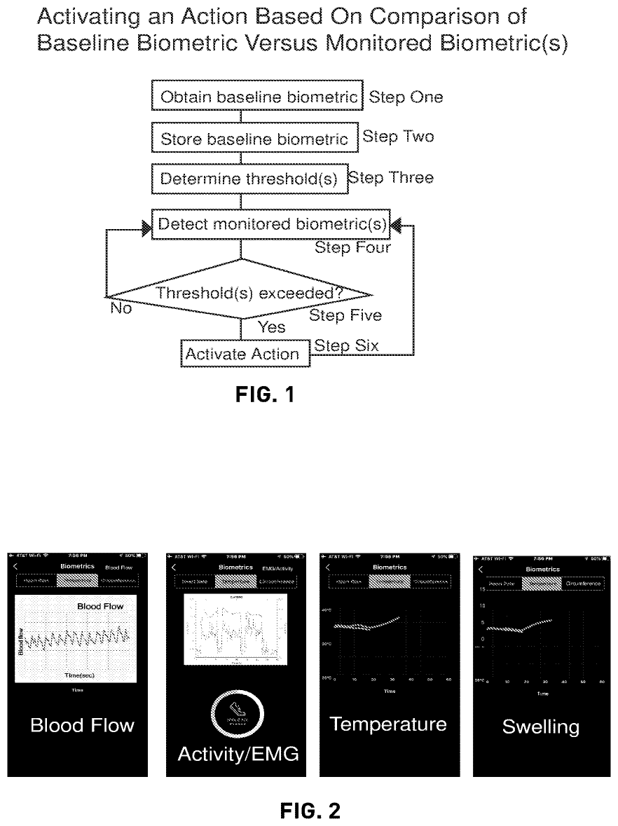

FIG. 1 is a flow chart illustrating an algorithm for activating an action based on comparison of a baseline biometric versus monitored biometrics.

For example, the baseline biometric can be obtained by a trained technician, nurse or doctor in a clinical setting with relatively expensive equipment and skilled labor, and the monitored biometrics can be obtained automatically, or semi-automatically, through the wearing of a suitable wearable electronic such as the wearable electronic described herein.

A wearable electronic monitors a change in a physical condition of a patient and compares it with a baseline biometric for that patient. As shown in the flowchart in FIG. 1(a), an initial baseline biometric data is obtained (step 1) and stored in a memory (step 2). The initial baseline biometric data is obtained using a baseline biometric test. For example, for a thrombotic condition, the baseline biometric data can be obtained at a clinic or hospital using relatively more sophisticated and accurate doppler ultrasound to measure blood through the deep veins of a patient. Other tests, such as D-dimer blood test, measuring a thrombosis biomarker in the blood, etc., can be used in combination or alone, to obtain baseline biometric data for the particular patient. This stored baseline data that is specific for the patient is used to determine at least one patient-specific threshold for one or more monitored biometric parameters (step 3). That is, the patient-specific thresholds are adjusted depending on an accurately measured baseline condition of the patient and will thus be dependent on the stored baseline biometric data. For example, if the blood flow measurement and/or the D-dimer blood test shows the patient is currently susceptible to a thrombotic event, then the thresholds will likely be lower before an action is activated such as a warning notification or change in an applied electroceutical treatment.

The one or more monitored biometric parameters are detected using biometric sensors (step 4). As represented by the GUI screens shown in FIG. 2, the biometric sensors can detect the classic symptoms of an impending thrombotic condition or other relevant biometric, including, but not limited to, a change in surface vessel blood flow, a lack of detected patient activity, a change in skin temperature or swelling. These detected biometrics can be particularly useful if biometric sensors are worn on both legs of a patient and a comparison is made of changes to one leg versus the other. The monitored biometric parameters are dependent on at least one physiological change of a patient occurring after the baseline biometric data is obtained and occurs in response to at least one of a therapeutic treatment, a change in a health related condition and a progression of a disease. At least one exceeded threshold is determined dependent on the detected one or more monitored biometric parameters and the at least one patient-specific threshold (step 5). If a threshold has been exceed, at least one action is activated depending on the determined exceeded threshold (step 6). The at least one action is at least one of a notification to a patient, a change in the therapeutic treatment, and a notification to at least one trusted receiver. The action may also be de-identified collection of biometric data for cloud-based storage and Big Data analysis.

FIG. 1 illustrates an algorithm for activating an action based on a comparison of a patient's baseline biometric versus monitored biometrics. The baseline biometric is typically obtained by a trained healthcare professional in a clinical setting using specialized equipment, while the monitored biometrics can be obtained automatically or semi-automatically through a wearable electronic device.

The algorithm begins by obtaining and storing the initial baseline biometric data using a baseline biometric test (step 1 and 2). The patient-specific thresholds for one or more monitored biometric parameters are then determined based on the stored baseline biometric data (step 3). The thresholds are adjusted depending on the accurately measured baseline condition of the patient, making them specific to the individual.

The monitored biometric parameters are detected using biometric sensors (step 4), and may include classic symptoms of a particular condition or disease, such as a change in blood flow, lack of activity, or changes in skin temperature or swelling. These parameters are dependent on physiological changes occurring after the baseline biometric data is obtained and occur in response to a therapeutic treatment, a change in a health-related condition, or the progression of a disease.

If an exceeded threshold is detected based on the monitored biometric parameters and the patient-specific thresholds (step 5), at least one action is activated (step 6). The action may include a notification to the patient, a change in the therapeutic treatment, a notification to a trusted receiver, or de-identified collection of biometric data for cloud-based storage and analysis.

The algorithm allows for personalized monitoring and intervention based on a patient's unique baseline biometric data and specific health conditions.

FIG. 2 shows smartphone graphical user interface screens of monitored biometrics.

For example, if there is a sudden increase in temperature of one leg versus the other, and at the same time, swelling is detected in the leg that is also undergoing the rise in skin temperature, these combined exceeded thresholds can be used to trigger a more urgent response, such as a red flag alert to the patient, a family member and a trusted receiver, such as the patient's healthcare provider. If no threshold is exceeded, the process flow of detecting the monitored biometrics continues until a threshold is exceeded. If the action is activated because a threshold is exceeded, the threshold levels can be automatically adjusted to created a higher degree of sensitivity to a monitored biometric change. That is, the monitored biometrics can be compared over time so that the monitoring of the patient is continuously adjusted depending on feedback from the patient's own body in response to an applied treatment (electroceutical, mechanical or pharmaceutical) and/or the disease progression.

The notification to the trusted receiver may include suggested diagnosis and current treatment options determined through an AI-powered web crawler. In this case, as an example, a doctor who is monitoring more than one patient using the inventive remote patient monitoring system can have a dashboard provided where each patient's condition is continuously updated depending on the monitored biometrics for the patient. If a change in condition is indicated by the monitored biometrics, an AI-powered web crawler can be employed to provide the doctor with the latest standard of care or other relevant data pulled in from online sources (see, for example, Devi R S, Manjula D, Siddharth R K. An Efficient Approach for Web Indexing of Big Data through Hyperlinks in Web Crawling. ScientificWorldJournal. 2015; 2015:739286. doi: 10.1155/2015/739286, which is incorporated by reference herein in its entirety).

In the case of DVT, the applied treatment may include at least one of an applied electroceutical treatment for activating a muscle pump of the patient and a pharmaceutical treatment for treating a cardiovascular condition. The at least one physiological change may include an indication of a change in the cardiovascular condition. The at least one action includes transmitting an alert, modifying the therapeutic treatment, and transmitting data dependent on at least one of the at least one physiological change, the one or more biometric parameters, the therapeutic treatment and the least one patient-specific threshold

The step of determining the at least one patient-specific threshold (step 3) may include determining from a data set of the one or more monitored biometric parameters whether the data set is acceptable for deciding that the at least one physiological change threshold has been exceeded. The step of determining the at least one patient-specific threshold may further comprise applying a statistical weighting to each of the one or more monitored biometric parameters, where the statistical weighting is dependent on a predetermined value of a ranking of importance in detecting each of the at least one physiological change for said each of the one or more monitored biometric parameters relative to others of the one or more monitored biometric parameters.

FIG. 2 shows several screen shots of a smartphone App user interface displaying the monitored biometrics obtained through the wearable electronic device described in the invention. These biometrics can include temperature, blood flow, activity, and swelling, among others, and can be monitored for both legs of a patient to compare changes in one leg versus the other.

If any of the monitored biometrics exceed the patient-specific threshold, an action is triggered, which may include sending a notification to the patient, a family member, and/or a trusted healthcare provider. The notification may also include suggested diagnosis and treatment options based on AI-powered web crawling of relevant online sources.

In the case of DVT, the applied treatments may include electroceutical or pharmaceutical treatments, and the monitored biometrics may indicate changes in cardiovascular conditions. The patient-specific threshold is determined based on a data set of the monitored biometrics, and statistical weighting can be applied to each biometric parameter based on the importance of detecting each physiological change relative to others.

The continuous monitoring of the patient allows for adjustments to be made in real-time, providing a more sensitive response to changes in the patient's condition. This invention can be used in various medical and non-medical applications, including remote patient monitoring, personal fitness tracking, and workplace safety monitoring, among others.

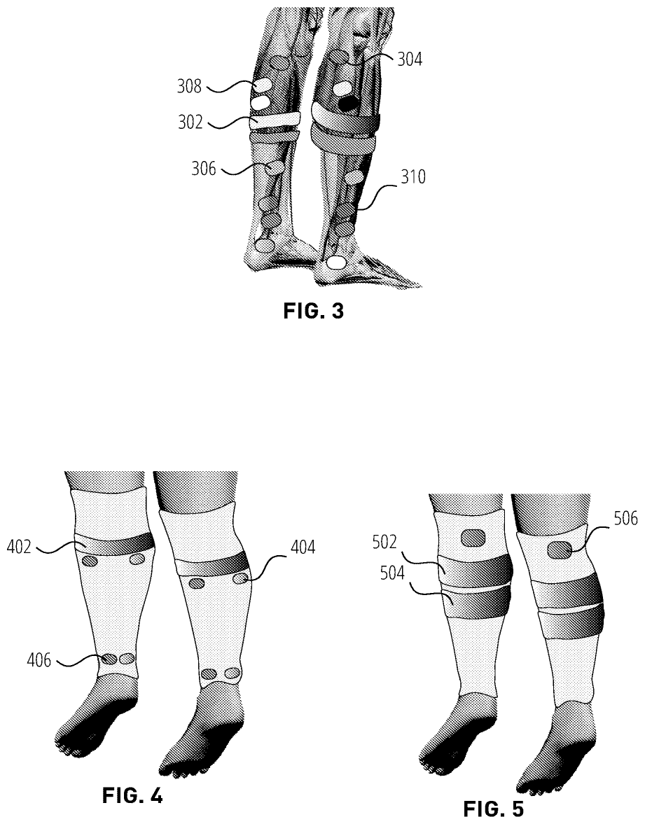

FIG. 3 illustrates the location of various biometric detectors, sensors, transmitters, processors, actuators, etc., on the lower legs of a patient. The system comprises actuators 302, detectors 304, processors 306, sensors 308, and transmitters 310 applied to the body depending on the sensor and the detected biometric. For example, on the lower legs the actuators 302 can include skin contract electrodes that actuate the muscles of the patient through electrical muscle stimulation applied through the skin. The detectors 304 can include blood flow detectors that detect blood flowing through surface or deep veins. Processors 306 can include a microcontroller and multiplexing circuitry to route electrical signals to and from the skin contact electrodes so that both EMS and EMG can be applied/detected through the same electrodes. Sensors 308 can include skin temperature sensors that measure a change in skin temperature on each leg and at various positions on the leg. Transmitters 310 can include a bluetooth transmitted associated with the microcontroller for transmitting raw data or an analyzed test result to a remote interface, such as a smartphone APP.

Using the detected biometric data, an analysis can be made of a therapeutic effect based on an activated physiological change and one or more detected biometric parameters. For example, the activated physiological change can be involuntary muscle contractions in response to an applied EMS. One detected biometric parameter can be the detection of a coagulation cascade factor present in the sweat or blood of the patient. Another detected biometric parameter may be the detection of blood flow through blood vessels in a body part, such as the legs. Another detected biometric parameter may be a change in the circumference of the leg caused by edema. Another detected biometric parameter may be a change in skin temperature caused by the onset of a thrombotic condition. Other biometric parameters are listed and described elsewhere. In accordance with an exemplary embodiment, the therapeutic effect may be the result of an administered therapeutic, such as a pharmaceutical therapy, e.g., an anticoagulant and/or an electroceutical therapy, e.g., EMS applied to activate the muscle pump. In addition, or as an alternative, to analyzing a therapeutic effect, disease progression or modification can be determined by monitoring the one or more detected biometric parameters.

The choice of the one or more detected biometric parameters may depend on the physiological condition, disease, fitness level, treatment being monitored, or other use case for the analysis of the therapeutic effect. Biometric parameters can be detected as an alternative or in addition to the ones described herein. For example, the biometric detection of biomarkers, such as thrombin and/or d-dimer, other proteins, other chemical, electrical or movement related biomarkers, may be used for treatment and monitoring of conditions related to the contact system for coagulation and inflammation, including DVT and/or VTE.

In accordance with a non-limiting embodiment of the invention, the blood flow information obtained by a blood flow detector can be used to detect a change in blood viscosity that may indicate a concerning condition such as thickening of the blood due to thrombotic conditions. The volume of blood detected and blood flow measurements can be used, along with, for example, the relative concentration of red blood cells compared to other blood constituents to check for a change in viscosity due to a thrombotic event as opposed to a change in viscosity due to a change in hydration (e.g., if the patient consumes water and/or receives an intravenous saline drip). Since the viscosity will change depending on hydration, a detected change in blood viscosity might not indicate thickening or thinning of blood due to coagulation tendency changes. By detecting a value for the concentration of red blood cells (for example, by detecting a change in reflected red light from the blood vessel) and normalizing for a given volume, then it is possible to calculate a value compared to a threshold (which may be obtained from a baseline patient reading) to indicate that the blood is becoming more or less stickier or thicker not due to hydration changes but instead the presence of potential clot forming coagulation factors. In the case of a disease, such as Covid-19, this stickiness of the blood can be an important determination of the prognosis of the patient and any remedial treatments needed to prevent blood clots from becoming a life or limb threatening condition.

FIG. 4 illustrates an embodiment of an electroceutical combination treatment device for applying an electroceutical signal in combination with detecting a biometric physiological response. The system comprises swelling detector 402, swelling detector 404, and sensors 406. EMS electrodes apply electrical signals to the body. The same electrodes can also be used as EMG electrodes to detect electrical signals from the body. An electronic circuit is connected with the wearable electronic garment. Depending on the intended use, the EMS electrodes can be used for EMG or other signal detection so that bi-directional electrical signals are applied through a plurality of individually addressable electrodes routed through an electrode multiplex circuit and a signal multiplex circuit for applying a sequential EMS signal and detecting biometric feedback, for example, from the calf of a patient. In accordance with an aspect of the invention, a digital therapeutic device garment is provided with a plurality of individually addressable electrodes supported by the garment for applying a sequential EMS signal and detecting biometric feedback from the calf of a patient. The individually addressable electrodes are for at least one of applying stimulation electrical signals to the skin of a patient and detecting biometric electrical signals from the skin of the patient. At least one of a signal detector for detecting the biometric electrical signals and a signal generator for generating the stimulation electrical signals are provided. An electrode multiplex circuit addresses the plurality of individually addressable electrodes by at least one of routing the biometric electrical signals from the skin of the patient through more than one of the plurality of individually addressable electrodes to the signal detector and routing the stimulation electrical signals from the signal generator through more than one of the plurality of individually addressable electrodes to the skin of the patient. A microprocessor controls the signal detector, the signal generator, the electrode multiplex circuit and other circuit components.

An embodiment can be configured as a pair of comfortable, washable, stockings that detect early physiological changes indicating thrombotic conditions, and apply electrical stimulation to automatically activate the muscle pump to help return blood flow towards the heart. Early signs of VTE may be detected days or even weeks before a patient would normally be prompted to seek medical advice. In this example, a smartphone is used to control the electronics and for a graphical user interface. The acquired biometric data is relayed through the smartphone, or directly from the wearable electronic device, to an access point and on to cloud storage connected to, for example, the internet, an insurance company or government server farm, or a hospital's intranet.

The embodiment can include push button control located on the electronics and/or be controlled by a smartphone APP. The patient's muscle pump EMS treatment can be applied automatically, with automatic modification to intensity, duration and other characteristics of the applied EMS signal that depends on the detected biometric parameters (for example, so that the duration and intensity are only as much as necessary).

A microprocessor can control the electrode multiplex circuit to route the biometric electrical signals from the skin of the patient sequentially through more than one of the plurality of individually addressable electrodes to the signal detector. In accordance with this embodiment, a single EMS signal source can service multiple individually addressable electrodes with the EMS signal routed as desired for an intended therapy, such as for the sequential squeezing of the deep veins in the legs to promote blood flow in the direction back to the heart.

This same circuitry, or something similar, can also be used to route signals from multiple sensors. For example, the sensors may be the same type, such as a solid-state temperature or IR-heat sensor, to enable multiple sensors to feed in and be sampled from, a single electronic detection circuit. The sensors can be different types, for example, a varying-resistance circumference sensor and a solid-state temperature sensor can have their signal output conditioned so that the same circuitry can be used for a number of signal acquisition, transmission and storage schemes. Other signal routing and transmission functionality can be similar multiplexed, enabling, for example, one communication channel to service many sensors. These features enable a reduction in the circuit complexity needed onboard the wearable electronic garment and/or enhance the functionality and features. For example, for privacy concerns it may be most advantageous to compress, filter, analyze, store and/or encrypt data as close as possible to the data source. In this case, the garment may have onboard electronics to perform these functions. Or, some other mix of electronic features and functions may be preferable. For example, raw data may be transmitted direct from the wearable electronic garment to be relayed by an access point or smartphone and then compressed, filtered, analyzed, stored and/or encrypted, etc., at a cloud-based server.

In FIG. 4 and FIG. 5, an electroceutical combination treatment device is shown, which applies an electroceutical signal in combination with detecting a biometric physiological response. The system includes various components such as swelling detectors 402 and 404, sensors 406, and EMS electrodes that apply electrical signals to the body. The EMS electrodes can also be used as EMG electrodes to detect electrical signals from the body. An electronic circuit is connected with the wearable electronic garment, and depending on the intended use, the EMS electrodes can be used for EMG or other signal detection so that bi-directional electrical signals are applied through a plurality of individually addressable electrodes routed through an electrode multiplex circuit and a signal multiplex circuit for applying a sequential EMS signal and detecting biometric feedback.

An embodiment of the electroceutical combination treatment device can be configured as a pair of comfortable, washable stockings that detect early physiological changes indicating thrombotic conditions and apply electrical stimulation to activate the muscle pump automatically to help return blood flow towards the heart. A smartphone can be used to control the electronics and provide a graphical user interface. The acquired biometric data is relayed through the smartphone, or directly from the wearable electronic device, to an access point and on to cloud storage connected to, for example, the internet, an insurance company or government server farm, or a hospital's intranet.

The system can be controlled via push button control located on the electronics and/or by a smartphone APP. The patient's muscle pump EMS treatment can be applied automatically, with automatic modification to intensity, duration and other characteristics of the applied EMS signal that depend on the detected biometric parameters. A microprocessor can control the electrode multiplex circuit to route the biometric electrical signals from the skin of the patient sequentially through more than one of the plurality of individually addressable electrodes to the signal detector.

The circuitry used in the electroceutical combination treatment device can also be used to route signals from multiple sensors. This same circuitry can be used for a number of signal acquisition, transmission, and storage schemes, enabling a reduction in the circuit complexity needed onboard the wearable electronic garment and/or enhancing the functionality and features. Raw data may be transmitted directly from the wearable electronic garment to be relayed by an access point or smartphone and then compressed, filtered, analyzed, stored and/or encrypted, etc., at a cloud-based server, or onboard electronics on the garment may perform these functions.

FIG. 5 illustrates an embodiment of an electroceutical treatment device for monitoring physiological changes in response to an administered treatment. The system comprises skin contact electrodes 502, 504, and sensors 506. The embodiment is an example of of an electroceutical combination treatment device for monitoring physiological changes in response to an administered pharmaceutical and/or electroceutical treatment and/or disease progression. The biometric parameters may include a strain gauge formed from an elastic resistance strip that reversibly changes a detectable resistance value based on being stretched. This provides a circumference detector that can monitor a change in the swelling in the lower leg.

The wearable electronic stockings shown in FIG. 5 are designed to monitor physiological changes in response to an administered treatment. The skin contact electrodes 502 and 504 are used to apply EMS signals to the skin of the patient, while also detecting biometric electrical signals from the skin of the patient. The sensors 506 are used to monitor other biometric parameters, including a strain gauge formed from an elastic resistance strip that can detect changes in the circumference of the lower leg, indicating swelling.

This embodiment is an example of an electroceutical combination treatment device for monitoring physiological changes in response to an administered treatment, whether it be a pharmaceutical and/or electroceutical treatment and/or disease progression. The skin contact electrodes can be used to apply electrical stimulation to activate the muscle pump to help return blood flow towards the heart, and can also be used to detect electrical signals from the body, such as EMG signals.

The electrodes and sensors are connected to an electronic circuit that is controlled by a microprocessor, allowing for automatic modification of the intensity, duration, and other characteristics of the EMS signal depending on the detected biometric parameters. This circuit can also route the biometric electrical signals from the skin of the patient sequentially through more than one of the plurality of individually addressable electrodes to the signal detector, allowing for multiple biometric parameters to be monitored simultaneously.

This embodiment can be used in combination with a smartphone or other device for remote monitoring and control, allowing for real-time monitoring of the patient's biometric parameters and treatment response. The acquired biometric data can be relayed through the smartphone or other device, and on to cloud storage for further analysis and monitoring.

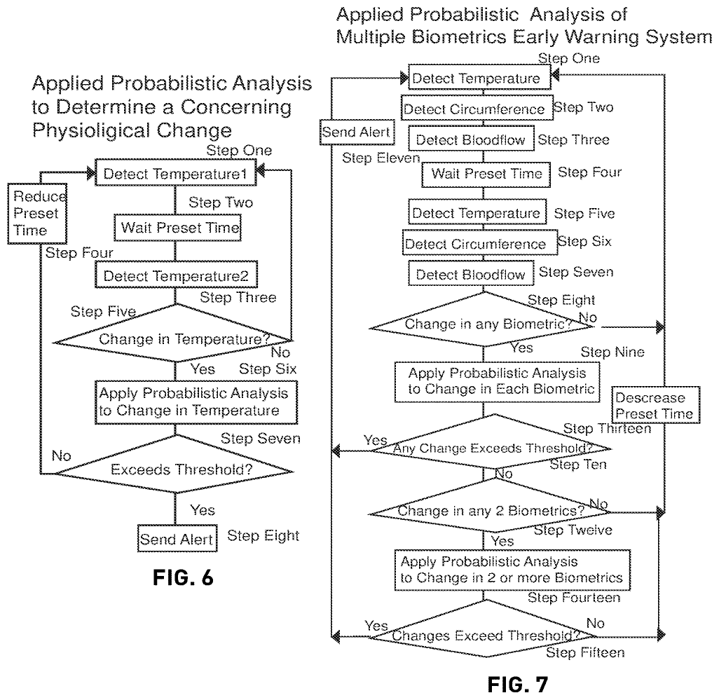

FIG. 6 is a flow chart illustrating an algorithm for applied probabilistic analysis to determine a concerning physiological change.

As a simple, low cost wearable electronic early warning system, a first biometric value can be compared to a second biometric value to note a physiological change indicating that an at-risk patient may be undergoing a concerning condition, such as a thrombotic event. A patch, ring, bracelet, anklet, sock, belt, or other wearable electronic can be constructed to automatically detect one or more biometric parameters indicative of a physiological change indicating the start of a concerning condition detectable at a body part such as the legs, wrist, foot, torso, neck, etc. In an embodiment shown herein, the wearable electronic is configured as a wrap, but other form factors are also contemplated, including a compression stocking, sock, sleeve, or cuff.

The embodiments described herein and others can be used to obtain copious and specific biometric data from a human or animal body for training artificial intelligence and machine learning (AI/ML) type of algorithms. This collection of biometric data from large numbers of individuals with similar physical conditions can be used by artificial intelligence algorithms using Big Data analysis to improve the software and hardware of the wearable electronic. The analysis of the detected biometric parameter can be used to automatically adjust an applied therapy for the individual patient and also to improve the application of treatment to all users of the wearable electronic through automatic updates to the software controller the applied therapy. AI/ML refers herein to various Big Data analysis techniques where large datasets of information is used to train an algorithm to identify patterns or other useful information contained in the Big Data.

A first biometric reading is taken (Step One). After a preset time (Step Two) a second biometric reading is taken (Step Three). The biometric readings are compared to see if there has been any change in the biometric reading occurring over time (Step Four). Probabilistic analysis is applied to determine if the change exceeds a threshold (Step Five) and if so, then an alert is sent (Step Six). If the change does not exceed a threshold (Step Six) then the preset time can be reduced (optionally, Step Seven) so that the biometric readings are detected and compared with a longer interval between samples (Steps One, Two and Three). Any of the sensors, detectors, and/or biometrics and biomarkers described herein or others available or detectors not specified can be utilized for obtaining the detected biometrics described in this flow chart and other flowcharts discussed herein.

Probabilistic analysis can be applied to this wearable electronic digital healthcare device by using statistical models to evaluate the likelihood of a concerning condition based on changes in biometric readings over time. For example, the device can be programmed to calculate the probability of a thrombotic event based on the change in biometric parameters such as heart rate, blood pressure, and oxygen saturation levels.

The probabilistic analysis can be based on historical data from previous patients or population studies, which can be used to develop predictive models for detecting early warning signs of a concerning condition. The device can also incorporate machine learning algorithms that learn from real-time data and adjust the probabilistic models accordingly.

The threshold for triggering an alert can be set based on the probabilistic analysis, with a higher threshold indicating a higher probability of a concerning condition. The threshold can be adjusted based on factors such as the patient's age, medical history, and lifestyle.

Probabilistic analysis can enhance the accuracy and reliability of the wearable electronic digital healthcare device as an early warning system by providing a more objective and quantitative evaluation of changes in biometric parameters over time. This can lead to earlier detection of concerning conditions, which can improve patient outcomes and reduce healthcare costs.

Probabilistic analysis can be applied to the device by using statistical models to evaluate the likelihood of a concerning condition based on changes in biometric readings over time. The device can be programmed to calculate the probability of a concerning condition, such as a thrombotic event, based on the change in biometric parameters such as heart rate, blood pressure, and oxygen saturation levels. Historical data from previous patients or population studies can be used to develop predictive models for detecting early warning signs of a concerning condition. The device can also incorporate machine learning algorithms that learn from real-time data and adjust the probabilistic models accordingly.

The threshold for triggering an alert can be set based on the probabilistic analysis, with a higher threshold indicating a higher probability of a concerning condition. The threshold can be adjusted based on factors such as the patient's age, medical history, and lifestyle. By using probabilistic analysis, the wearable electronic digital healthcare device can provide a more objective and quantitative evaluation of changes in biometric parameters over time, leading to earlier detection of concerning conditions, improved patient outcomes, and reduced healthcare costs.

FIG. 7 is a flow chart illustrating an algorithm for an early warning system with applied probabilistic analysis of multiple biometric parameters.

First biometric1, biometric2 through biometricN readings are taken (Step One, Two, Three). After a preset time (Step Four) a second biometric1, biometric2 through biometricN readings are taken (Step Five, Six, Seven). If there has been a change in any of the detected biometrics over the preset time (Step Eight), then probabilistic analysis is applied to the detected change in each biometric (Step Nine). If any analyzed change exceeds a threshold, which is predetermined or calculated for each type of biometric reading (Step Ten), then an alert is sent (Step Eleven) and process flow returns to detect the biometric1, biometric2 through biometricN readings (Step One, etc.). If the change threshold of none of the detected biometrics has been exceeded (Step Ten), then it is determined if there was any change (e.g., below the individual change threshold) in two or more biometrics over the preset time (Step Twelve). If there was a change in two or more biometrics (Step Twelve) then probabilistic analysis is applied to the change in the two or more biometrics (Step Fourteen) and if that analysis indicates that the changes exceed a threshold (Step Fifteen), then an alert is sent (Step Eleven) and process flow continues to Step One. If there was not a change in any biometric over the preset time (Step Eight), then process flow continues to detect the biometric1, biometric2 through biometricN again (Steps One, Two, Three). If there wasn't a change in two or more biometrics (Step Twelve) or the analyzed changes did not exceed the threshold (Step Fifteen), then to increase detection frequency or to conserve battery power, consumables such as sweat stimulation chemicals, and data collection memory and transmission, the preset time can be changed (Step Thirteen) depending on a desired increase or decrease in detection, etc., and process flow continues to Step One. Any of the sensor, detectors, and/or biometric and biomarkers described herein or others available or detected but not specified can be utilized as the detected biometric described in this flow chart and other flowcharts discussed herein. As described herein, the algorithm described in any of the flowcharts herein can be utilized in a wearable electronic that monitors a change in a physical condition of a patient and compares it with a baseline biometric for that patient.

FIG. 7 is a flow chart illustrating an algorithm for an early warning system with applied probabilistic analysis of multiple biometric parameters.

Any of the sensors, detectors, and/or biometrics and biomarkers described herein or others available or detected but not specified can be utilized as the detected biometric described in this flow chart and other flowcharts discussed herein. As described herein, the algorithm described in any of the flowcharts herein can be utilized in a wearable electronic that monitors a change in a physical condition of a patient and compares it with a baseline biometric for that patient.

The flow chart represents an algorithm for an early warning system that uses multiple biometric parameters to detect and alert concerning changes in the body. By monitoring multiple biometric parameters, the accuracy and reliability of the analysis can be improved, and the thresholds for each biometric can be tailored to the individual patient.

The use of multiple biometric parameters also provides a more comprehensive view of the patient's physiological state, which can improve the accuracy of the analysis. For example, if one biometric reading falls within the normal range, but another biometric reading indicates a concerning change, the system can still trigger an alert based on the overall pattern of changes.

Moreover, over time, data collected from a number of patients in the same patient population can be used to refine the probabilistic models used in the analysis. By incorporating data from a larger patient population, the algorithm can become more accurate in predicting the likelihood of a concerning condition based on changes in multiple biometric parameters.

The algorithm applies probabilistic analysis to each detected change in the biometric parameters. The threshold for triggering an alert is predetermined or calculated for each type of biometric reading, and alerts are sent if any analyzed change exceeds the threshold.

If there is no change in any biometric parameter over the preset time, the process continues to detect the biometric readings again. If there is a change in any biometric parameter, the algorithm applies probabilistic analysis to the detected changes in each biometric parameter. If the analyzed changes exceed the threshold for any of the biometric parameters, an alert is triggered. If the analyzed changes do not exceed the threshold for any of the biometric parameters, the algorithm determines if there was a change in two or more biometric parameters over the preset time. If there was a change in two or more biometric parameters, the algorithm applies probabilistic analysis to the change in the two or more biometric parameters. If the analyzed changes exceed the threshold, an alert is triggered.

The use of multiple biometric parameters and applied probabilistic analysis in the algorithm can enhance the accuracy and reliability of the wearable electronic digital healthcare device as an early warning system. The system can detect and alert concerning changes in the body at an early stage, allowing for earlier intervention and improved patient outcomes. Additionally, by incorporating data from a larger patient population, the algorithm can become more accurate over time, further improving patient care.

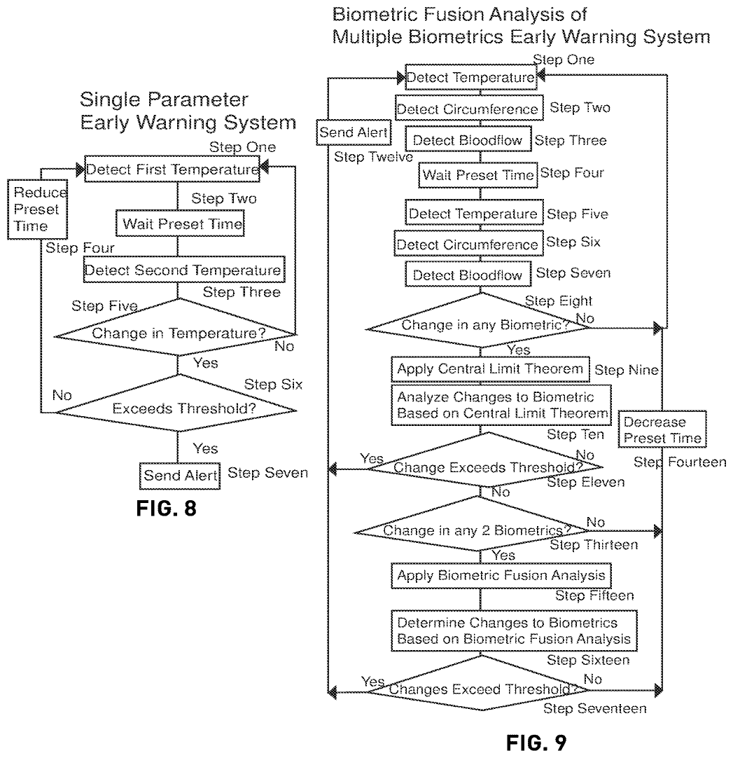

FIG. 8 is a flow chart illustrating an algorithm for a single parameter early warning system.

As a simple, low cost wearable electronic early warning system, a first biometric value, such as temperature can be compared to a second biometric value to note a physiological change indicating that an at-risk patient may be undergoing a concerning condition, such as a thrombotic event.

A first temperature reading is taken (Step One). After a preset time (Step Two) a second temperature reading is taken (Step Three). The temperature readings are compared to see if there has been any change in the temperature reading occurring over time (Step Four), if the change exceeds an alert-threshold (Step Five) then an alert is sent (Step Six). If the change doesn't exceed the alert-threshold but does exceed an “all-is-well” threshold (Step Six) then the preset time can be reduced (optionally, Step Seven) so that the temperature readings are detected and compared sooner (Steps One, Two and Three). Any of the sensors, detectors, and/or biometric and biomarkers described herein or others available or detected but not specified can be utilized to obtain the detected biometric described in this flow chart and other flowcharts discussed herein.

Since patients will likely have two legs that will not likely undergo a simultaneous thrombotic condition, such as the slowing of blood through the deep veins caused by a forming blood clot, the two legs can be used for comparison with each other through a logic flow similar to those described herein. For example, if one leg experiences an increase in blood flow as compared to the other leg (or the rate of increase or decrease of temperature of the two legs is different), there is a potential that there is a blood clot forming in the warmer leg.

Probabilistic analysis can be applied to this scenario by using statistical models to evaluate the probability of a thrombotic event based on changes in temperature readings over time. The device can be programmed to calculate the probability of a thrombotic event based on the change in temperature between the first and second readings, as well as the rate of change in temperature over time.

The probabilistic analysis can be based on historical data from previous patients or population studies, which can be used to develop predictive models for detecting early warning signs of a thrombotic event. The device can also incorporate machine learning algorithms that learn from real-time data and adjust the probabilistic models accordingly.

The threshold for triggering an alert can be set based on the probabilistic analysis, with a higher threshold indicating a higher probability of a thrombotic event. The threshold can be adjusted based on factors such as the patient's age, medical history, and lifestyle.

To further enhance the accuracy and reliability of the device, the two legs can be used for comparison with each other, as described in the scenario. This can help to detect subtle differences in temperature readings between the legs, which may indicate the formation of a blood clot in one leg.

Probabilistic analysis is applied to this scenario by using statistical models to evaluate the likelihood of a thrombotic event based on changes in temperature readings over time. The device can be programmed to calculate the probability of a thrombotic event based on the change in temperature between the first and second readings, as well as the rate of change in temperature over time.

To enhance the accuracy and reliability of the device, the two legs can be used for comparison with each other, as described in the scenario. This can help to detect subtle differences in temperature readings between the legs, which may indicate the formation of a blood clot in one leg.

The threshold for triggering an alert can be set based on the probabilistic analysis, with a higher threshold indicating a higher probability of a thrombotic event. The threshold can be adjusted based on factors such as the patient's age, medical history, and lifestyle.

Probabilistic analysis can also be based on historical data from previous patients or population studies, which can be used to develop predictive models for detecting early warning signs of a thrombotic event. The device can incorporate machine learning algorithms that learn from real-time data and adjust the probabilistic models accordingly.

The single parameter early warning system can be used as a simple and low-cost way to monitor for early warning signs of a thrombotic event. By incorporating probabilistic analysis and using historical data and machine learning algorithms, the accuracy and reliability of the device can be further improved.

FIG. 9 is a flow chart illustrating an algorithm for biometric fusion analysis of multiple biometrics to determine a physiological change.

The algorithm can be used to enable artificial intelligence pattern recognition to improve the hardware, software and use of a wearable electronic digital therapeutic device that includes a memory for storing baseline biometric data, biometric sensors for detecting monitored biometric parameters, a processor for determining patient-specific thresholds and exceeded thresholds, and an action module for activating at least one action depending on the determined exceeded threshold, such as a notification to a patient, a change in therapeutic treatment, or a notification to a trusted receiver. The device is designed to monitor physiological changes in response to therapeutic treatment, changes in health conditions, and disease progression, and to tailor the treatment plan based on patient-specific thresholds for monitored biometric parameters.

Biometric fusion is the use of multiple types of biometric data and/or methods of processing the data, to improve the performance of biometric systems. For example, a type of fusion is score-level fusion, which is the combination of matcher scores to improve accuracy. The scores used in fusion can be obtained through the use of multiple types of data for each subject and is typically applied for identification (such as face and fingerprint, or fingerprints from different fingers). In accordance with the inventive wearable electronic digital therapeutic device and applications described herein, biometric fusion analysis is used to improve the accuracy and effectiveness of using detected biometrics for patient therapeutic, monitoring and diagnostic applications. The power of probabilistic analysis, such as biometric fusion analysis can be employed in a machine learning technique to provide predictive outcome determination (e.g., is there a thrombotic condition that should trigger an alert) using one or both the copious amount of biometric data obtainable via the present invention for the individual patient and/or the aggregate of such data obtained from many patient-user's and/or comparisons with data from healthy individuals and/or physiological computer models. Machine learning techniques typically utilize large data sets and improve through the accumulation of confirmation of predictions and analysis modification interactions, such data and analysis made easily available using the present invention.

A first biometric1 (e.g., temperature), biometic2 (e.g., circumference) through biometricN (e.g., blood flow) readings are taken (Step One, Two, Three). After a preset time (Step Four) a second biometric1, biometic2 through biometricN readings are taken (Step Five, Six, Seven). The first and second biometrics are compared and if there has been a change exceeding a predetermined threshold in any of the detected biometrics over the preset time (Step Eight), then probabilistic analysis, such as an application of the central limit theorem or other statistical analytic model, is applied (Step Nine) to analyze the detected change in each biometric (Step Ten). If the analyzed change exceeds a threshold, which is predetermined or calculated for each type of biometric reading (Step Eleven), then an alert is sent (Step Twelve) and process flow returns to detect the biometric1, biometic2 through biometricN readings (Step One, etc.). In a single biometric change determination, if the change threshold of none of the detected biometrics has been exceeded (Step Eleven), then it is determined if there was a change in two or more biometrics over the preset time (Step Thirteen), in this case the change threshold may be lower than the change threshold used for the single biometric change determination. In a multiple biometric change determination, if there was a change in two or more biometrics (Step Thirteen) then probabilistic analysis, such as biometric fusion analysis, is applied to the change in the two or more biometrics (Step Fifteen) and decision trigger changes to biometrics based on the biometric fusion analysis are determined (Step Sixteen). That is, the type of statistical analysis used, the thresholds, preset time, etc., can be systematically modified so that over time, a better predictive result is obtained for the system used by the individual patient, and/or in the aggregate, the device itself is improved for the patient category.

If analysis indicates that the changes exceed an alert threshold (Step Seven), then an alert is sent (Step Twelve) and process flow continues to Step One. If there was not a change in any biometric over the preset time (Step Eight), then process flow continues to detect the biometric1, biometic2 through biometricN again (Steps One, Two, Three). If there wasn't a change that exceeds the thresholds (Step Eleven) (Step Seventeen), then to conserve battery power, consumables such as sweat stimulation chemicals or the body's ability to generate sweat on demand, and data collection memory and transmission, the preset time can be decreased (Step Fourteen) and process flow continues to Step One.

The inventive embodiment is part of a wearable electronic digital therapeutic product platform that enables effective drug/device combination therapies, electroceutical therapy, and biometric data acquisition and analysis. A use-case for this platform is for cardiovascular diseases, such as venous thrombosis (VTE).

In accordance with an embodiment, the wearable electronic monitors a change in a physical condition of a patient and compares it with a baseline biometric for that patient. A wearable electronic monitors a change in a physical condition of a patient and compares it with a baseline biometric for that patient. Initial baseline biometric data is stored in a memory. The initial baseline biometric data is obtained using a baseline biometric test. At least one patient-specific threshold is determined for one or more monitored biometric parameters dependent on the stored baseline biometric data. The one or more monitored biometric parameters are detected using biometric sensors. The monitored biometric parameters are dependent on at least one physiological change of a patient occurring after the baseline biometric data is obtained and occurs in response to at least one of a therapeutic treatment, a change in a health related condition and a progression of a disease. At least one exceeded threshold is determined dependent on the detected one or more monitored biometric parameters and the at least one patient-specific threshold. At least one action is activated depending on the determined exceeded threshold. The at least one action is at least one of a notification to a patient, a change in the therapeutic treatment, and a notification to at least one trusted receiver.

The notification to the trusted receiver may include suggested diagnosis and current treatment options determined through an AI-powered web crawler. The applied treatment may include at least one of an applied electroceutical treatment for activating a muscle pump of the patient and a pharmaceutical treatment for treating a health related condition, such as a cardiovascular condition. The at least one physiological change may include an indication of a change in the cardiovascular condition. The at least one action includes transmitting an alert, modifying the therapeutic treatment, and transmitting data dependent on at least one of the at least one physiological change, the one or more biometric parameters, the therapeutic treatment and the least one patient-specific threshold.

The step of determining the at least one patient-specific threshold comprises determining from a data set of the one or more monitored biometric parameters whether the data set is acceptable for deciding that the at least one physiological change threshold has been exceeded. The step of determining the at least one patient-specific threshold may further comprise applying a statistical weighting to each of the one or more monitored biometric parameters, where the statistical weighting is dependent on a predetermined value of a ranking of importance in detecting each of the at least one physiological change for said each of the one or more monitored biometric parameters relative to others of the one or more monitored biometric parameters.

Biometric fusion analysis can be used to improve patient outcomes by combining multiple types of biometric data and methods of processing the data to enhance the performance of biometric systems. By using biometric fusion analysis, wearable electronic devices can provide a more comprehensive and accurate assessment of a patient's physiological condition, leading to earlier detection and more effective treatment of medical conditions.

One example of biometric fusion analysis is score-level fusion, which involves combining matcher scores to improve accuracy. In the context of wearable electronic devices, biometric fusion analysis can involve the combination of multiple biometric parameters, such as temperature, circumference, and blood flow, to provide a more robust and accurate assessment of a patient's condition.

To apply biometric fusion analysis, the wearable electronic device can take multiple biometric readings, as described in the scenario, and compare them to determine if there has been a change exceeding a predetermined threshold over a preset time. If a change is detected, probabilistic analysis, such as the central limit theorem or other statistical analytic models, can be applied to the change in each biometric. If the change exceeds a threshold, an alert is sent.

In the case of multiple biometric changes, biometric fusion analysis can be applied to determine if the changes indicate a concerning condition. This can involve using machine learning techniques to analyze the relationship between the different biometric parameters and how they change over time. By combining the data from multiple biometric parameters, biometric fusion analysis can provide a more accurate assessment of a patient's physiological condition, leading to more effective treatment and better patient outcomes.

In addition to improving the accuracy and effectiveness of biometric monitoring, wearable electronic devices can also be used to customize treatment based on patient-specific thresholds. The device can determine at least one patient-specific threshold for one or more monitored biometric parameters, which can be used to customize the treatment plan based on the individual needs of the patient.

Biometric fusion analysis can improve the performance of wearable electronic devices for therapeutic, monitoring, and diagnostic applications, leading to better patient outcomes and more efficient healthcare delivery.

Biometric fusion analysis is a powerful technique that can be used to combine multiple types of biometric data and methods of processing the data to improve the performance of biometric systems. One of the main advantages of using biometric fusion analysis in conjunction with the wearable electronic digital therapeutic device and applications described in this patent application is that it can provide a more comprehensive and accurate assessment of a patient's physiological condition. By combining data from multiple biomarkers, wearable electronic devices can detect early warning signs of concerning changes in a patient's condition, leading to earlier detection and more effective treatment of medical conditions.

In addition to improving the accuracy of biometric monitoring, biometric fusion analysis can also be used to customize treatment based on patient-specific thresholds. By determining patient-specific thresholds for monitored biometric parameters, the wearable electronic device can tailor the treatment plan to the individual needs of the patient, leading to better patient outcomes.

Furthermore, the power of probabilistic analysis, such as biometric fusion analysis, can be employed in machine learning techniques to provide predictive outcome determination. This means that the device can learn from real-time data and adjust the probabilistic models accordingly, improving over time to provide better results for the individual patient, and in the aggregate, improving the device itself for the patient category. By using machine learning techniques, the device can analyze the copious amounts of biometric data obtained from each patient and aggregate data from many patients to develop more accurate predictive models for detecting early warning signs of medical conditions. This can lead to earlier detection and more effective treatment, improving patient outcomes and reducing healthcare costs.

Another advantage of biometric fusion analysis is that it allows for more personalized and targeted treatment plans. By combining data from multiple biometric parameters, wearable electronic devices can provide a more complete picture of a patient's physiological condition and help healthcare providers develop more targeted and effective treatment plans. This can lead to better patient outcomes and reduced healthcare costs by avoiding unnecessary treatments or procedures.

The use of biometric fusion analysis in conjunction with wearable electronic devices has the potential to revolutionize healthcare delivery by providing more accurate and comprehensive assessments of a patient's physiological condition. By combining multiple biometric parameters and employing probabilistic analysis and machine learning techniques, wearable electronic devices can provide earlier detection and more effective treatment of medical conditions, leading to better patient outcomes and reduced healthcare costs.

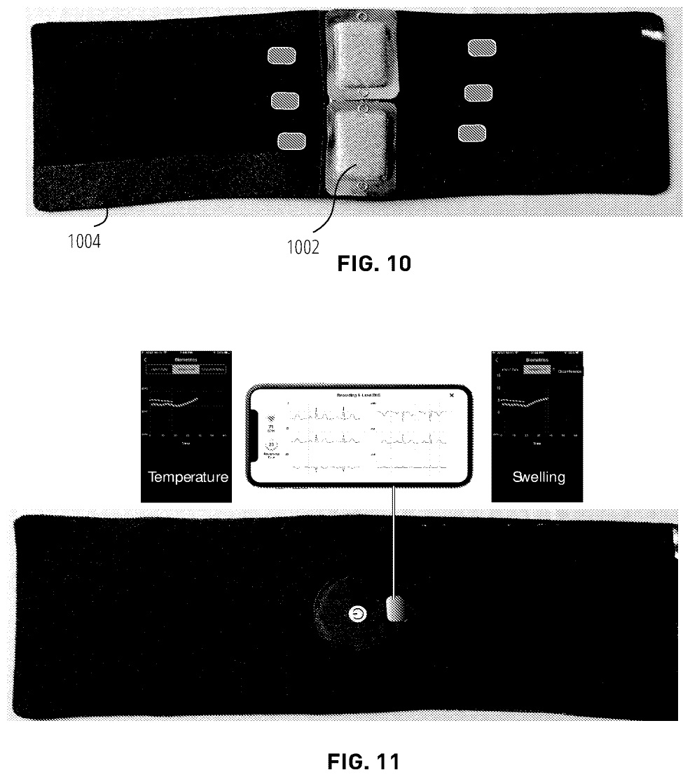

FIG. 10 is a photo showing an embodiment of a wearable electronic for DVT prevention and remote patient monitoring. The system comprises an electrodes 1002 and a garment 1004. A relatively simple embodiment is configured as a wrap for applying electrical muscle stimulation (EMS) as an electroceutical therapy, which may be applied as an alternative to, or complementary to, the use of an anticoagulant or other pharmaceutical treatment.

A pair of electrodes apply an EMS signal through the skin at the calf of a patient inducing involuntary contractions in the muscles adjacent to the deep veins. The involuntary muscle contractions induce a squeezing action on the deep veins and promote a flow of blood through the veins in a direction towards a heart of the patient.

A simple modular system can be provided that is mobile, washable, low cost, and designed for scalable manufacturing. The components include: electrodes/carrier (moisture-holding electrodes for home use, gel electrodes for clinical use); EMS generator electronics (wirelessly connected to a smartphone and/or network); and wearable electronic garment (wrap, sleeve or stocking).

The EMS signal generating electronics are controlled by a smartphone APP with a user-friendly interface, with large text size and limited but meaningful information. The smartphone APP graphical user interface (GUI) is used to select automatic or manual control of the muscle pump mode, TENS pain mitigation treatment modes, and several massage modes. The APP also collects usage data and provides a history of the device use and selected settings. The embodiment includes the features of automatic 30-minute muscle pump treatment, massage and TENS pain relief modes, can use both gel electrodes and moisture-holding electrodes, smartphone APP control and user interface, modular system designed for ease of use, low cost, and scalable manufacturing.

FIG. 11 illustrates the wearable electronic for DVT prevention with biometric sensors for EMG and EKG, temperature and swelling detection.

The wearable electronic can be configured as the wrap, as shown, or another garment configuration such as a sleeve or stocking. This embodiment includes a combination of electroceutical therapy (muscle pump activation) and biometric detection and data acquisition.

The detected biometric parameter(s) change depending on disease progression, the bioactive action of a drug and/or the applied electroceutical therapy. A microprocessor analyzes the detected biometrics and automatically modifies the application of the electroceutical treatment in response to the detected biometric signal.

There is a growing list of detectable biometric parameters, the selection of which depends on factors such as cost and use-case. Examples include skin temperature, skin color, blood flow, pulse, heartbeat, blood pressure, blood viscosity, skin tightness/swelling, blood chemistry, sweat chemistry, electronic biomarker, chemical biomarker, and electromyography. Other suitable biometric or environmental condition can also be detected including ambient temperature, time of day, GPS location, etc.

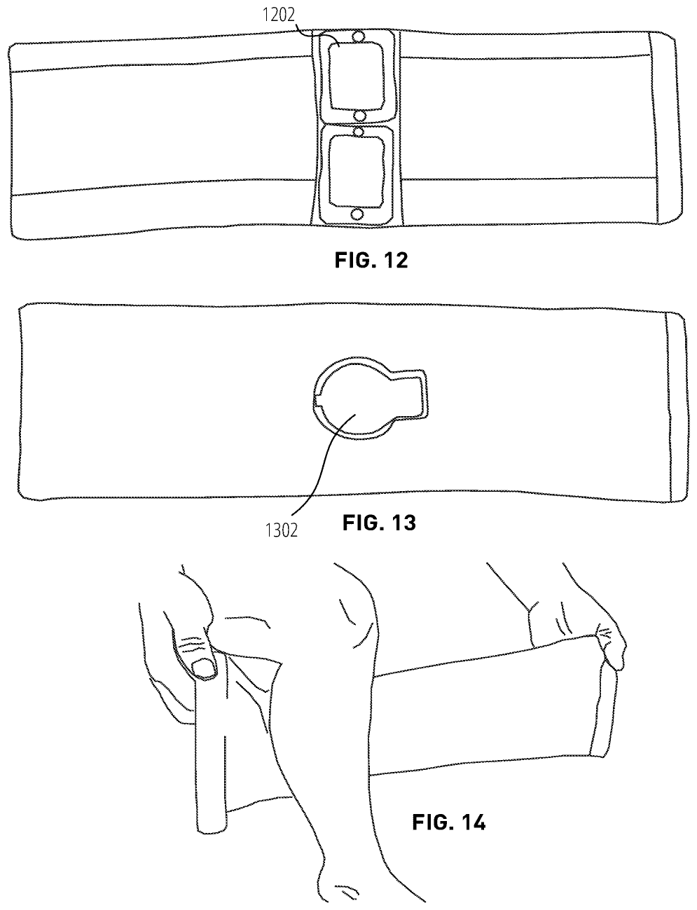

FIG. 12 is a drawing showing a wrap embodiment with foam fit electrodes for applying an EMS signal and receiving EMG, heart, or other electrical biometric signals from the same electrodes. The wrap includes EMG and EMS detecting/applying electrodes 1202.

A network connection, direct from the wearable electronic device through a wireless Internet access point or via a smartphone relay, can collect and distribute the patient data using the cloud. It should be noted that the use of cloud storage for the collection, analysis and storage of patient data is currently a frontier of the global healthcare system. Integrity of the data, long-term storage, and especially privacy concerns must be addressed before the potential of biometric data to improve global health can be realized (see, Blockchain: A Panacea for Healthcare Cloud-Based Data Security and Privacy?, Esposito et al., IEEE Cloud Computing, January/February 2018).

The embodiment is the scaffolding to acquire useful biometric information and apply electroceutical therapies. The use of the embodiment with a wireless network connection enables secure and accurate open-source or privately controlled access to a vast amount of collected biometric data to researchers and HCPs around the world.

FIG. 13 is a drawing showing removable electronics disposed on the wrap embodiment. The wrap includes electronics 1302.

The need to create a secure system that ensures privacy and integrity of the data is an active research area. For example, cryptographic techniques are being explored to ensure data confidentiality and privacy. Access control mechanisms (e.g., password, biometric and other access control tools) have been suggested to limit and control who can access patient data (see, Alam et al., “A Cross Tenant Access Control (CTAC) Model for Cloud Computing: Formal Specification and Verification,” IEEE Transactions on Information Forensics and Security, vol. 12, no. 6, 2017, pp. 1259-1268). The use of both access control and cryptographic techniques, such as attribute-based encryption has also been suggested (see, M. Li et al., “Scalable and Secure Sharing of Personal Health Records in Cloud Computing Using Attribute-Based Encryption,” IEEE Transactions).

With smart solutions conforming to new standards and regulations, the future of global health will surely include the widespread collection and analysis of biometric data.

However, when many people are wearing some form of biometric detection technology every day (for example, the embodiment or a “smart” T-shirt), it will not be efficient to put online every heartbeat, so some filtering and compression of collected data is needed. The biometric data can be filtered to detect anomalies or potential anomalies in the collected biometric data. There also needs to be a data privacy and security layer as close to the source (the user or patient) as possible.

FIG. 14 is a drawing illustrating the placement of the wrap embodiment onto the lower leg of a patient.

These embodiments are part of a medical device product platform that uses wearable electronics, blockchain and AI to collects biometric data, such as heartbeat and sweat chemistry from a living organism, a human, a pet or livestock, anonymously and securely store that data using blockchain technology and use AI to look for patterns in the data to determine health aspects of the population such as heart disease and diabetes.

The embodiment can be used to acquire useful biometric information and apply electroceutical therapies. The use of the embodiment with a wireless network connection enables secure and accurate open-source or privately controlled access to a vast amount of collected biometric data to researchers and HCPs around the world. This biometric data may also become part of the patient's permanent Electronic Health Record (EHR). An EHR is the systematized collection of patient and population electronically-stored health information in a digital format. These records can be shared across different health care settings

The need to create a secure system that ensures privacy and integrity of the data is an active research area. For example, cryptographic techniques are being explored to ensure data confidentiality and privacy (see, S. Nepal et al., “Trustworthy Processing of Healthcare Big Data in Hybrid Clouds,” IEEE Cloud Computing, vol. 2, no. 2, 2015, pp. 78-84.; G. S. Poh et al., “Searchable Symmetric Encryption: Designs and Challenges,” ACM Computing Surveys, vol. 50, no. 3, 2017). Access control mechanisms (e.g., password, biometric and other access control tools) have been suggested to limit and control who can access patient data (see, Alam et al., “A Cross Tenant Access Control (CTAC) Model for Cloud Computing: Formal Specification and Verification,” IEEE Transactions on Information Forensics and Security, vol. 12, no. 6, 2017, pp. 1259-1268.). The use of both access control and cryptographic techniques, such as attribute-based encryption has also been suggested (see, M. Li et al., “Scalable and Secure Sharing of Personal Health Records in Cloud Computing Using Attribute-Based Encryption,” IEEE Transactions). With smart solutions conforming to new standards and regulations, the future of global health will surely include the widespread collection and analysis of biometric data.

However, when many people are wearing some form of biometric detection technology every day (for example, the embodiment or a “smart” T-shirt), it will not be efficient to put online every heartbeat, so some filtering and compression of collected data is needed. The biometric data can be filtered to detect anomalies or potential anomalies in the collected biometric data. There also needs to be a data privacy and security layer as close to the source (the user or patient) as possible.

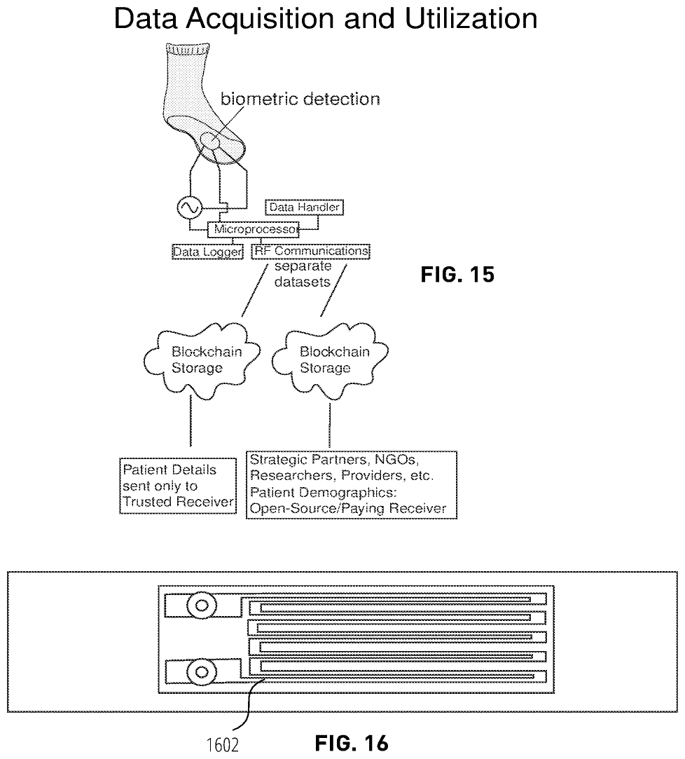

FIG. 15 is a block diagram showing dual data paths for privacy ensured data acquisition and utilization.

The algorithm of this flow chart can be used to provide dual paths of data transmitted for a wearable electronic garment that can monitor biometric data relevant to deep vein thrombosis (VTE). The garment includes biometric sensors, a microprocessor to create separate patient-identifying and non-identifying data sets, a transmission module, an action module, and a muscle pump device to improve blood flow. The garment also includes features like smartphone app control, alerts, and reminders, and can use AI and ML algorithms to improve patient outcomes.

There can be considered two uses for the patient's biometric data: Patient monitoring for prevention and treatment; and Population studies to improve global healthcare.

As part of the wearable electronics technology platform, separately created and maintained data bases can be used, one focused on patient treatment the other focused on population studies. Within the hardware and software of the embodiment that is on the patient (as close to the data source as practical), a microprocessor can control a data handler to create two separate data sets of the collected biometric information.

One data set (HCP Dataset) includes the necessary identifying information so that the health care provider knows which patient the data belongs to, and so that the HW/SW can be utilized for patient-specific features such as alerts, automatic treatment control, pill reminders, etc. The other data set (Research Dataset) is formatted without any patient-identifying data.

The HCP Dataset enables vital biometric data to be transmitted to a trusted receiver through a highly secured verification process that meets or exceeds the requirements for a particular governmental jurisdiction, and that ensures the patient that their biometric data will become part of their EHR without undue exposure to privacy-related drawbacks. The software-enabled capabilities of the data acquisition and usage can change from country to country.

The Research Dataset only has the information that is necessary to identify the general demographics of the patient (age, gender, prior and current medical conditions), so that the acquired biometric data along with the general demographic data enables researchers to improve treatments, drug discovery, etc., but without exposing the patient to any potential of their identity ever being conveyed.

The wearable electronics platform can be used as a tool for clinical trials of pharmaceuticals. The intended use of the embodiment is to improve deep vein blood flow to reduce the risk of VTE and to provide a clinical trials tool for remote patient monitoring of biometrics indicative of the classic symptoms of VTE. For example, when testing the efficacy and safety of a new anticoagulant drug, the embodiment can use to both for the prevention of DVT and to acquire data related to how the body is reacting to the new drug. Clinical trial participants can be monitored at home without requiring cost of nurse or technician, with constant monitoring of selected biometrics important to the clinical trial.

The embodiment is a wearable electronic garment that is worn on the lower legs and applies electrical signals to activate the muscle pump of the calf muscles for improving blood flow through the deep veins. Biometrics consistent with VTE development are measured continuously. The biometric data is collected and stored in either local memory and/or cloud.

According to the US Centers for Disease Control and Prevention (CDC), common symptoms include: swelling in the foot, ankle or leg, usually on one side; cramping pain in the affected leg that usually begins in the calf; severe, unexplained pain in the foot and ankle; an area of skin that feels warmer than the skin on the surrounding areas; and skin over the affected area turning pale or a reddish or bluish color. Some or all of these classic symptoms can be sensed using the embodiment with the proper biometric sensors.

Blood flows more slowly in veins than in arteries, which is why thromboses occurs most often in the veins. The deep veins in the calf and thigh are most often affected by deep vein thrombosis. Blood returning from the legs travels mainly through the deep veins. The low pressure of the blood flow in the venous system varies from relatively high flow (during muscle contraction) to low flow, for example, when sedentary. Blood flow in the veins below the heart and is assisted back up to the heart by the muscle pump. Many veins are located in the muscles and the squeezing action of the muscles promotes the flow of blood through the veins. That is, movement of the legs caused by muscle contractions squeezes the veins, which pushes the blood toward the heart.

The wearable electronic can be configured as a clinical trial tool that can be used to continuously monitor the lower legs of the patient to provide data relevant to VTE for the clinical trial. As well as provide an early warning system for VTE conditions before a clot has blocked blood flow in the deep veins (so the patient can be brought in for a doctor's visit instead of going to the emergency room).

A usage log can give the patient/HCP a quick indication of how long and when the muscle pump feature was used. A simple medicine reminder screen lets the patient select the time for a gentle reminder to take their pill, and a simple confirmation of adherence. As with all the biometric and user interactions, this adherence data can be provided to the caregiver, the drug company and/or the insurance provider to help ensure constant treatment improvements. Again, the user interface offers an opportunity to encourage the patient to continue adherence noting the days of uninterrupted pill taking and can also include a 1-click Order option that pops up at the correct time to ensure the patient always has their monthly drug supply.

Some of the possible features of the embodiment include: massage and TENS pain relief modes; automatic 30 minute muscle pump treatment; customized calibration routine for each user (only needs to be done once); can use both gel electrodes (for in-hospital) and moisture-holding electrodes (for home); modular system allows wearable electronic garment to vary depending on use-case (in-home, active and mobile, in-hospital, sleep, work); smartphone APP control and user interface; alerts and Reminders provided by APP; Dr./Patient connectivity and remote patient monitoring; potential for biometric sensors that enable significant patient-outcome advantages as well as data collection.

As an example, multiple temperature sensors can create a heat map with the rise at each sensor being statistically weighted so that the local heat area (area nearest to the formation of thrombosis in the deep vein) can be deduced. An example temperature example sensor is the DS18B20 Programmable Resolution 1-Wire Digital Thermometer.

The dual transmission path for biometric data described in FIG. 15 allows for both patient monitoring and population studies to improve global healthcare. The use of AI and ML algorithms can ensure that the correct data is transmitted for each path. The HCP Dataset includes identifying information to allow for patient-specific features and is transmitted securely to a trusted receiver. The Research Dataset only includes general demographic information and is used for population studies, without exposing the patient's identity.

The wearable electronics platform can be used for clinical trials, allowing for remote patient monitoring of biometrics important to the trial. The wearable garment applies electrical signals to activate the muscle pump of the calf muscles to improve deep vein blood flow and continuously measure biometrics consistent with VTE development. The embodiment can also be used as an early warning system for VTE conditions before a clot has blocked blood flow in the deep veins.

Features of the embodiment include massage and TENS pain relief modes, automatic muscle pump treatment, customized calibration routines, and smartphone app control with alerts and reminders. The use of biometric sensors can provide significant patient-outcome advantages as well as data collection. For example, multiple temperature sensors can create a heat map to deduce the local heat area nearest to the formation of thrombosis in the deep vein.

The use of AI and ML algorithms can improve individual patient outcomes and improve the invention used by a patient population by ensuring that the correct data is transmitted for each path and continuously improving probabilistic models based on real-time data.

FIG. 16 shows a swelling detector made by printing stretchable conductive ink on a stretch fabric. The wearable electronic includes a swelling detector 1602. The swelling detector comprises a serpentine pattern of conductive ink.

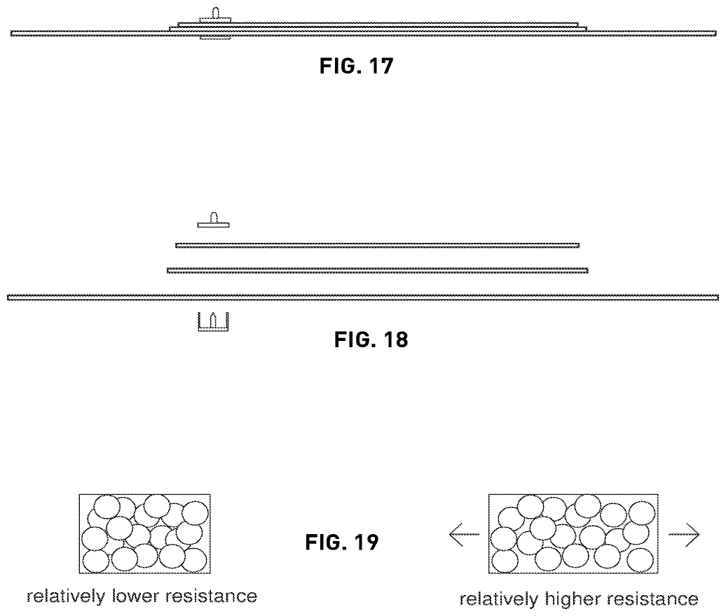

FIG. 17 is a side view of the swelling detector.

FIG. 18 is an exploded side view of the swelling detector.

FIG. 19 illustrates the detectable increase in resistance when conductive particulate of the swelling detector are separated as swelling occurs in a lower leg.

The inventive swelling detector is designed to detect changes in the circumference of a lower limb, which can be indicative of swelling. The detector is made by printing a serpentine pattern of stretchable conductive ink on a stretch fabric. The conductive ink includes conductive particles in a binder, which allows for the ink to stretch and deform with the fabric.

When the lower limb swells, the circumference increases, causing the distance between the conductive particles in the ink to increase. This separation between the conductive particles results in a measurable increase in resistance, which can be detected and used to indicate the presence of swelling.

The serpentine pattern of conductive ink is designed to increase the length of the conductive path and thus increase the sensitivity of the detector. This pattern also allows for the detector to stretch and deform with the fabric, ensuring that the device remains in contact with the skin and can accurately detect changes in the circumference of the limb.

The use of conductive ink and a stretchable fabric allows for the detector to be lightweight, flexible, and comfortable to wear for extended periods of time. Additionally, the use of this technology enables the detector to be printed on-demand, making it a cost-effective and scalable solution for monitoring swelling in patients with various medical conditions.

The inventive swelling detector provides a simple and effective solution for monitoring changes in the circumference of a lower limb, which can be indicative of swelling. This technology has the potential to improve the management and treatment of various medical conditions, including those related to cardiovascular disease, diabetes, and injury.

For detecting swelling, an all-elastic stretch sensor can be incorporated directly into the upper and lower bands of our wearable electronic VTE Prevention wrap. An example of a similar stretchable strain gauge has been demonstrated by the University of Houston, Highly Sensitive and Very Stretchable Strain Sensor Based on a Rubbery Semiconductor, ACS Appl. Mater. Interfaces, 2018, 10 (5), pp 5,000-5006, 2018. A stretchable strain sensor with printable components has recently been reported by the University of Florida, Highly Stretchable and Wearable Strain Sensor Based on Printable Carbon Nanotube Layers/Polydimethylsiloxane Composites with Adjustable Sensitivity, ACS Appl. Mater. Interfaces, 2018, 10 (8), pp 7,371-7380.

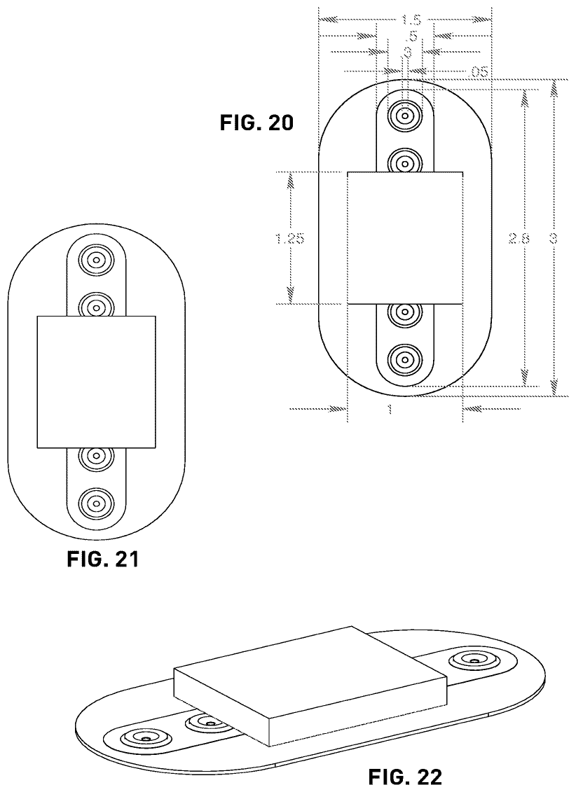

FIG. 20 is a top view showing the relative dimensions in inches of an embodiment of electronics with snap connectors for electrically and mechanically mounting on the wearable electronic.

FIG. 21 is a top view of the electronics.

FIG. 22 is a perspective view of the electronics.

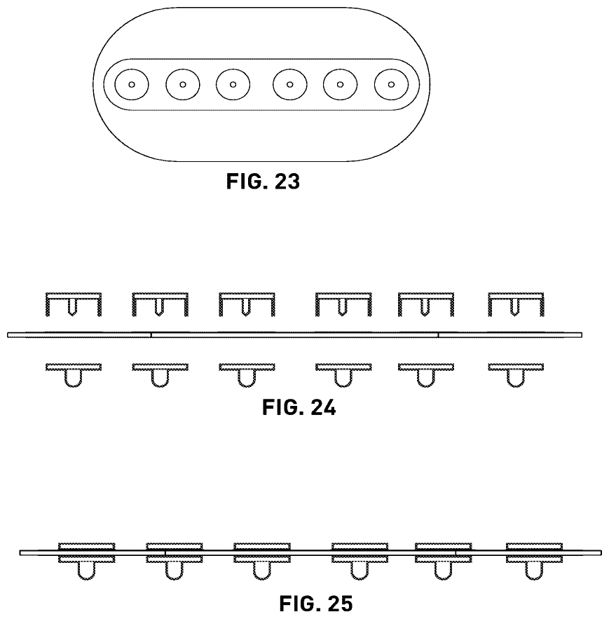

FIG. 23 shows a top view of electronics with multiple in-line snap connections

FIG. 24 is a side view showing snaps and flex circuit PCB before a crimping operation.

FIG. 25 is a side view showing snaps and flex circuit PCB after the crimping operation.

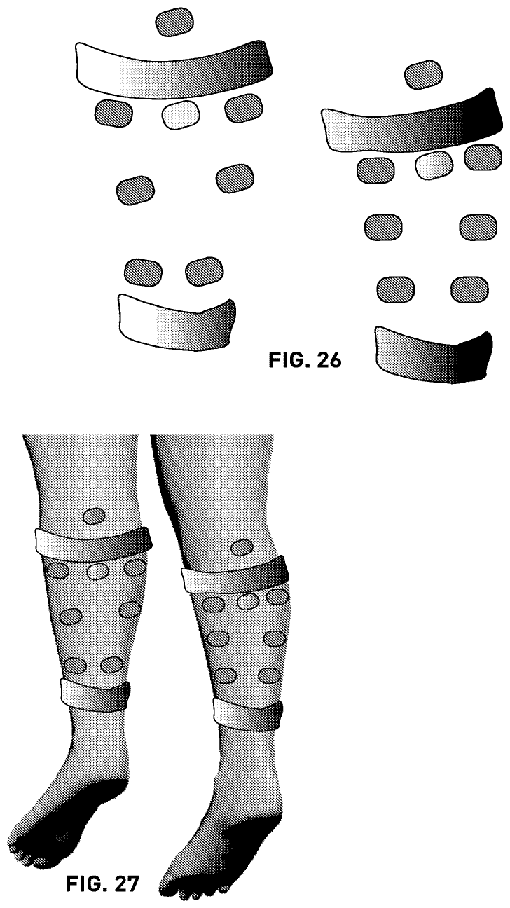

FIG. 26 shows multiple biosensors for detecting biometric parameters indicative of a thrombotic condition and/or diseases progression.

FIG. 27 shows the placement of biometric sensors on the lower legs of a patient.

A blood flow sensor from Kyocera is one of the smallest known optical blood-flow sensors, which measures the volume of blood flow in subcutaneous tissue. When light is reflected off blood within a blood vessel, the frequency of light varies—called a frequency or Doppler shift—according to the blood-flow velocity. This sensor utilizes the relative shift in frequency (which increases as blood flow accelerates) and the strength of the reflected light (which grows stronger when reflected off a greater volume of red blood cells) to measure blood-flow volume. The sensor is only 1 mm high, 1.6 mm long and 3.2 mm wide.

The Kardia Mobile ECG by AliveCor is an example of an ECG device with well-known electronics that can be modified to detect heartbeat and other heart related measurements. There are many small and inexpensive examples of blood pulse oximeters, automatic blood pressure readers, and skin temperature sensors that can be modified in accordance with the inventive digital therapeutic to detect temperature, blood pressure, pulse, blood oxygen and other related biometric parameters. The same electrodes that apply the EMS signal to activate the muscle pump also work with the AliveCor electronics to detect the heart rhythm. A third conductor, located on the electronics housing, is touched by the finger of the patient to enable a multi-lead EKG measurement.

EMG (electromyography) can be detected using the same EMS applying electrodes, indicating the patient's activation of the muscle pump, such as when walking. An accelerometer or GPS system can be used to provide an activity tracker.

Skin color can be detected using optical systems. Full-color skin imaging using RGB LED and floating lens in optical coherence tomography, disclosed by Yang B-W, Chen X-C, Full-color skin imaging using RGB LED and floating lens in optical coherence tomography, Biomedical Optics Express. 2010; 1 (5):1341-1346. doi: 10.1364/BOE.1.001341 shows an example of an LED based skin color sensor system that can be modified in accordance with the inventive digital therapeutic to detect skin color as a biometric parameter. It is noted that many of the various biometric detectors can share common components, reducing costs and enabling high speed sampling of different biometric parameters for the different exemplary embodiments described herein.

The sweat chemistry sensor may comprise a stretchable electrochemical sweat sensor made, for example, by the deposition of carbon nanotubes (CNTs) on top of patterned Au nanosheets (AuNS) as reported by the graduate school of converging science and technology, Korea University, Seoul (see, for example, Skin-Attachable, Stretchable Electrochemical Sweat Sensor for Glucose and pH Detection, ACS Applied Materials & Interfaces 2018 10 (16), 13729-13740 DOI: 10.1021/acsami.8b03342).

A smartphone APP can be used for the patient to manage the electronics(s) available on the wearable garments, to control and monitor the treatments, and also to capture the biometric data. After data authentication, the application reads the device data through an external framework such as provided through Continua, an industry organization of healthcare and technology companies dedicated to improving the quality of personal healthcare. Mobile Application screens provide an option to choose the different types of treatments with additional screens like History, Chat messenger, Reminders and so on. The application accommodates multiple platforms with great responsiveness for multiple resolutions. The application has a configuration set by the patient to control the paired devices with intensity and duration. The treatment instances held by the patient is tracked and may be shared with a remote HCP.

As the data is received by the mobile application from the wearable electronic, the data is stored into mobile local storage such as SQLite. The stored data can then be read by the application to perform the necessary action, and a synchronizer module taking care of processing the data to on onboard or cloud-based database. This ensures the application behaves consistently in offline mode with locally stored data.

An Admin application is a web portal having functionalities like registering users and user management with SMS and Email capabilities. In addition to Admin, a Provider application is also a web portal which has access to Patient related activities, report generation options and a chat feature. A proposed technology for the frontend web application is Angular 8 and for APIs, Dot net Core 3.1 The embodiment system also has the capability to send a SMS at the time of registration. An external tool can be used to facilitate this feature. The embodiment system can also have the email capability to send an invite at the time of registration and share the reports between the patient and HCP. The email communication can be enabled through third party delivery system such as SendGrid which is available on Azure. The system will also have the capability to send Notifications for users on performing specific activities in the system. A proposed Notification service is FCM (Firebase Cloud Messaging), which is a cloud service from Google.

Under the HIPAA regulations, cloud service providers (CSPs) such as AWS or Azure are considered business associates. The Business Associate Addendum (BAA) is a contract that is required under HIPAA rules to ensure that the cloud vendor appropriately safeguards protected health information (PHI). Almost all of the services that will be consumed in this project will fall under the HIPAA compliant category.

Azure enables us to address the implementation of technical, physical and administrative safeguards required by HIPAA. HIPAA compliance would be ensured when dealing with every aspect of the architecture.

The inventive digital therapeutic device and these example processes implemented as a software/hardware solution creates a drug/device combination therapy that puts the patient's own body into a real-time feedback loop. The embodiments described herein can be used for many types of diseases and conditions, and work with a large number of prescribed or over the counter drugs, herbal remedies, or other applications where an ingested chemical modifies a detectable biometric.

These therapies available through the inventive digital therapeutic device can be used as complementary or alternatives to drugs and surgery, and typically can typically continue for as long as the target drug is prescribed for the patient, and/or be employed before or after the prescribed drug is taken as treatment by the patient.

The data detection, transmission, and storage described herein provide a detailed history of the patient's adherence to the prescribed course of drug therapy. The biometric parameters such as those described herein with regards to the embodiments can also be detected, logged and/or transmitted, enabling a detailed history of the patient's therapy, course of treatment, measured results of treatment, etc., and can be made available to improve the care given to the particular patient, and in the aggregate, provide significant data along with that of other patients, to assist in new drug discovery, treatment modifications, and a number of other advantages of the beneficial cycle created by detection, transmission, storage and analysis of biometric data taken directly from the patient during the course of drug therapy and/or other treatments.

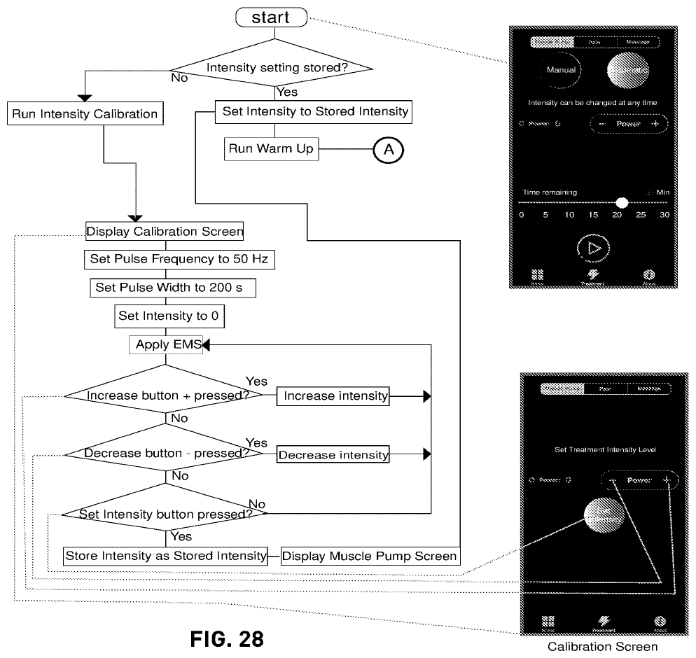

FIG. 28 is a flowchart illustrating an automatic muscle pump treatment algorithm.

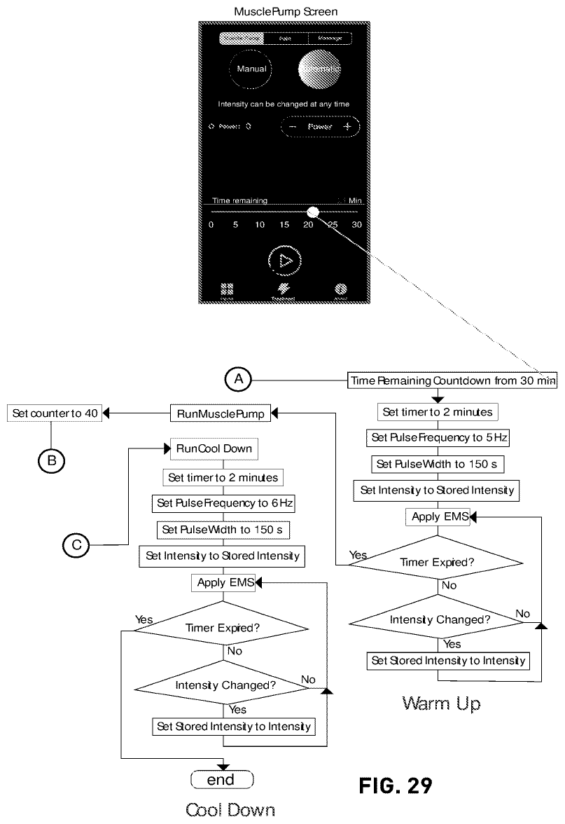

FIG. 29 continues the flowchart shown FIG. 28.

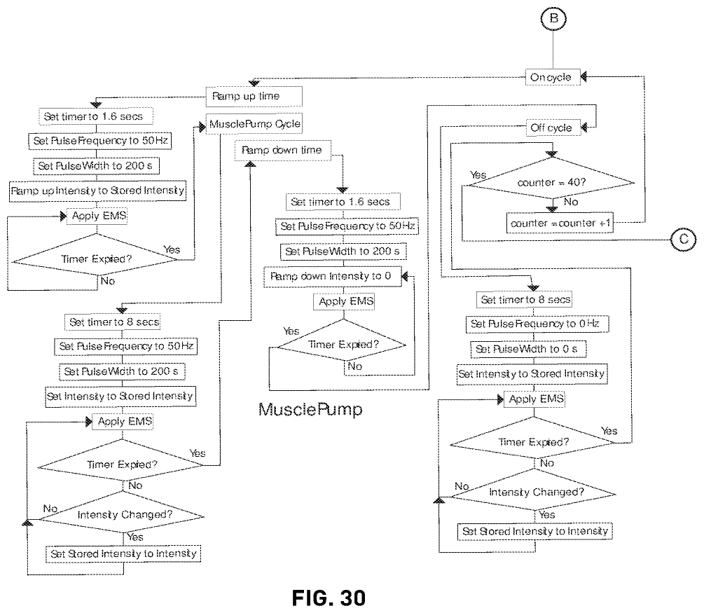

FIG. 30 continues the flowchart shown in FIG. 29.

This automatic treatment mode provides a patient with a convenient 30 minute treatment for activating the muscle pump via an applied EMS signal. The patient is able to adjust the intensity, even if the patient is using the Automatic mode. For example, if the patient becomes uncomfortable with the EMS power during the 30 minute automatic treatment, or the opposite, if the patient feels that a stronger intensity (power) would be better, instead of stopping the 30 minute treatment the patient has the option to increase or to lower the intensity (Power+ or Power−) and then set that new power level as the intensity for the remaining part of the 30 minute automatic treatment.

This is the same basic idea for the calibration routine. The first time the patient starts to use the DVT product in Automatic treatment mode the intensity (power) of the applied EMS signal must to be calibrated (set) for that patient. This is because the contractions caused by EMS signal should be strong but comfortable (i.e., the EMS power (intensity). But because every patient is different the applied signal must be calibrated with a custom maximum power setting for the patient. So, at least the first time the patient selects Automatic Treatment, the APP will take the patent through the calibration routine (the flowchart in FIG. 27(a)). Once the maximum power setting for the Automatic treatment is calibrated for the patient the electronics do not need to be calibrated again for that patient unless the patient chooses to calibrate again. However, the patient can choose to increase (Power+) or decrease (Power−) the EMS intensity if the patient becomes uncomfortable with the EMS power during the 30 minute automatic treatment, or the opposite, if the User feels that a stronger intensity (power) would be better.

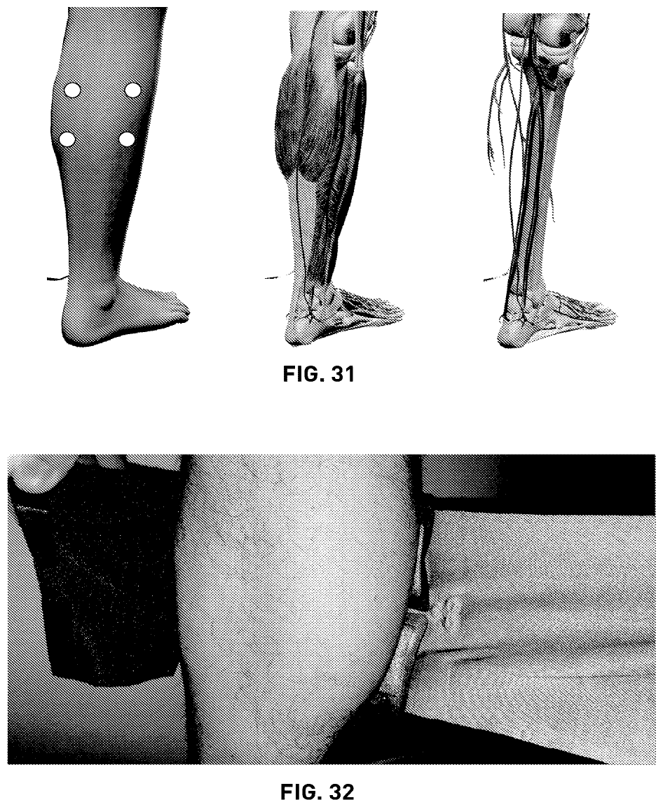

FIG. 31 shows the location of multiple temperature sensors for creating a heat map of a rise in temperature on the lower leg of a patient.

FIG. 32 shows a thermal harvesting construction of the wrap embodiment for DVT prevention with thermally conductive stretch fabric for conducting a temperature increase at one local area of the lower leg to a temperature sensor at another location.

In accordance with this embodiment, a weighted average of temperature rises can be used to help locate the closest skin surface area to an underlying clot formation. FIG. 29 shows a thermal harvesting construction of the wrap embodiment for DVT prevention with thermally conductive stretch fabric for conducting a temperature increase at one local area of the lower leg to a temperature sensor at another location. In this embodiment, the thermally conductive stretch fabric conducts heat from an area of the skin that locally warms up due to the formation of a clot in the deep veins of the lower legs. This embodiment might provide a lower cost solution that requires only one or a few temperature sensors to provide temperature rise detection over the larger surface of the back of the user's legs.

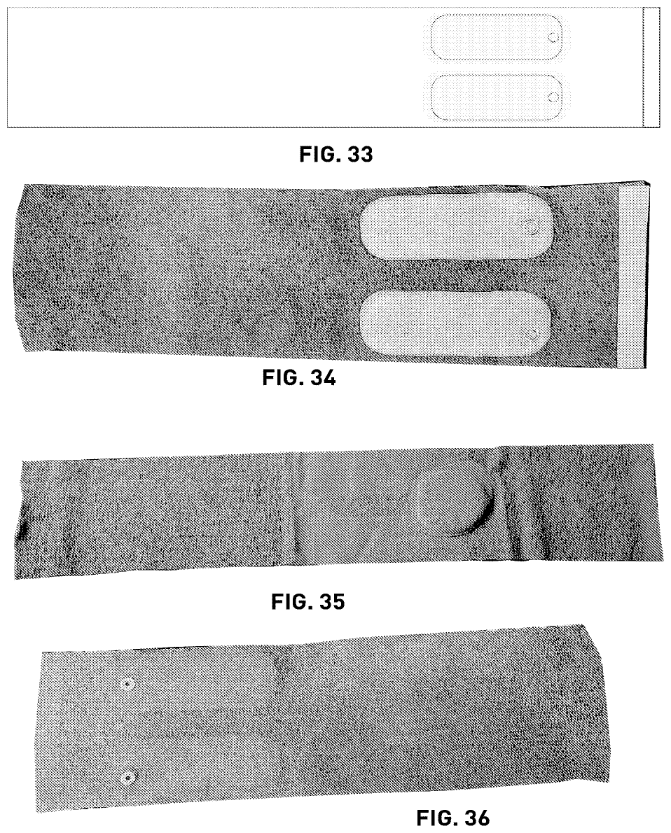

FIG. 33 illustrates a construction of a wearable electronic wrap having an elastic substrate that adheres to itself and not to skin, along with a skin adhesive strip for anchoring the substrate to facilitate wrapping around a body part.

FIG. 34 shows the wearable electronic wrap having two conductive stretch fabric skin contact electrodes.

FIG. 35 shows the wearable electronic wrap having a small, mobile, bluetooth enabled electronics and rechargeable battery package.

In accordance with this non-limiting exemplary embodiment, the wearable electronic comprises an elastic substrate that has a nonwoven material and elastic fibers. The substrate is cohesive to preferentially stick to itself and to not stick to skin of a user. Two or more skin contact electrodes are disposed on a top surface of the substrate. Snap connectors are provided for connecting the skin contact electrodes with an electrical signal generating circuit. A skin anchoring adhesive patch is disposed towards an end of the elastic substrate. The skin anchoring adhesive patch anchors the elastic substrate on a body part of the user while wrapping the elastic substrate around the body part.

FIG. 36 shows the wearable electronic wrap having conductive snaps for electrically communicating the skin contact electrodes with the electronics.

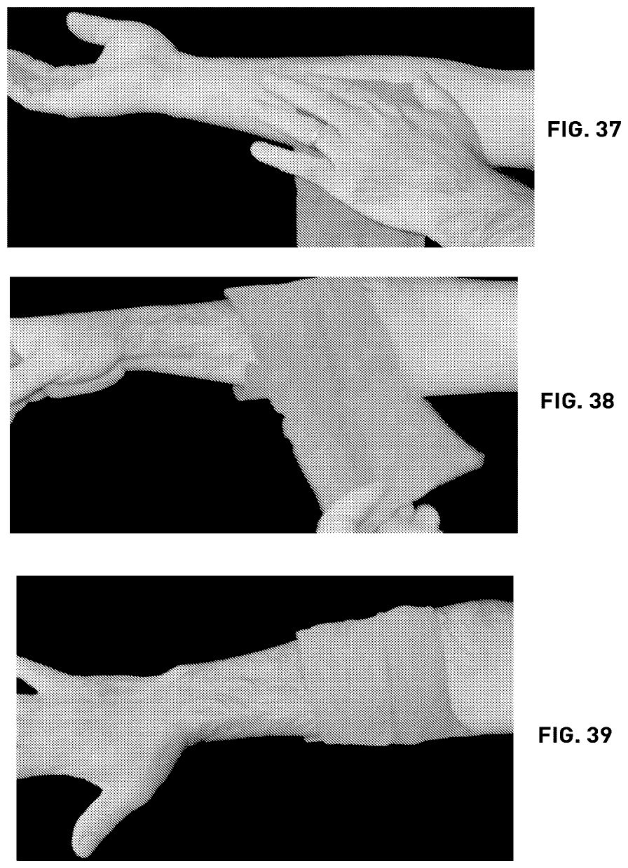

FIG. 37 shows the wearable electronic wrap having an end being anchored by the skin adhesive strip to the forearm of a user.

FIG. 38 shows the anchored wearable electronic wrap being wrapped using one hand of the user around the user's other forearm.

Wrapping the wearable electronic wrap can be especially difficult for a user that upper limb disability, so only has the use of one hand to perform the wrapping around the user's own body part, such as a lower leg or forearm. The skin adhesive strip provides a secure anchor for one end of the wrap. This makes completing an appropriately tight and consistent wrap around the body part much easier, especially for one handed wrapping.

FIG. 39 shows the wearable electronic wrap anchored around the forearm of the user.

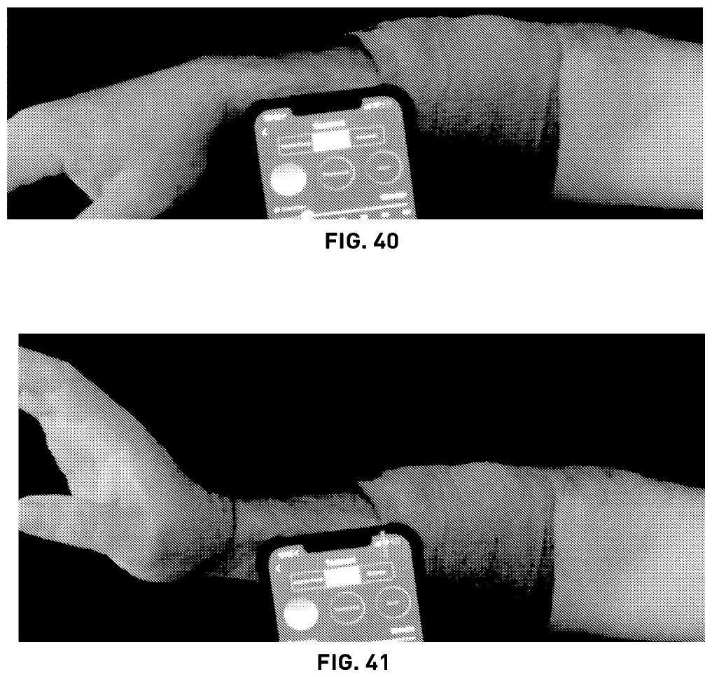

FIG. 40 shows the hand of the user in a resting position with an APP enabled smartphone used for controlling the bluetooth enabled electronics.

FIG. 41 shows the hand of the user pivoting at the wrist to stretch the muscles and tendons of the forearm to prevent contracture and atrophy.

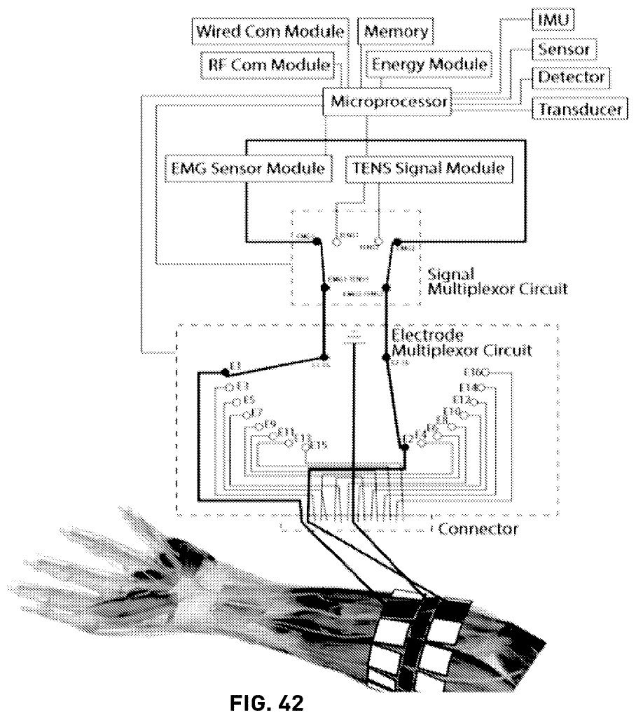

FIG. 42 illustrates an embodiment of the HHMI wearable electronic configured for upper limb disability recovery use-cases.

The wearable electronic can be designed as a wrap or sleeve that is easily put onto a patients limb or other body part. Described here is an exemplary embodiment of a wrap configured for upper limb disability recovery use-cases. The wrap or forearm sleeve is provided with skin contact electrodes for applying electrical signals causing involuntary muscle contractions in a stroke paralyzed limb. Other components may include, a Bluetooth module for wireless communication with an APP enabled smartphone, a wireless VR headset that provides audio and visual sensory cues, an onboard accelerometer, and a sweat chemistry sensor that generates a small amount of sweat on-demand and non-invasively identifies and monitors neurotropic chemical biomarkers that are present in sweat. The wearable electronic wrap is designed to prevent muscle atrophy and contracture and to help rebuild lost muscle memory and neural patterns. The VR training system provides immersive sensory experiences to aid in the rehabilitation process.

The hand of the user is shown in a resting position with an APP enabled smartphone used for controlling the bluetooth enabled electronics. The hand of the user pivots at the wrist to stretch the muscles and tendons of the forearm to prevent contracture and atrophy. In this use-case, the wearable electronic wrap is configured for use by a stroke survivor or other patient that has upper limb disability, including, but not to MS; Spinal Cord Injury; Brain Trauma; Cerebral Palsy; Parkinson's; Alzheimer's. Contracture and muscle atrophy are conditions that affect the upper limb of such patients. The wearable electronic wrap provides a simple, low cost solution to prevent muscle atrophy and contracture by applying EMS to the muscles of the upper forearm causing involuntary muscle contractions that pivot the hand at the wrist to stretch and maintain the condition of the muscle, tendons and ligaments. EMG can also be detected using the same or additional skin contact electrodes so that the progress of the patient and/or automatic treatment adjustment can be provided based on the EMG biometric feedback.

An exemplary use-case for a non-limiting embodiment is described herein for post-stroke recovery. Stroke is the greatest burden for the healthcare system among all neurological disorders and the leading cause of long term disability according to the WHO. In the US there are more than 4 million stroke survivors, two-thirds of them are currently disabled and receive rehabilitation services at hospital/clinics or at home. An embodiment of the HHMI wearable electronic can be configured for upper limb disability recovery use-cases. In accordance with an aspect of the invention, a comfortable wearable electronic garment is coupled with audio and video virtual reality to enhance rehabilitation progression and achieve better outcomes with respect to the standard of care.

The exemplary embodiment of the wearable electronics and can be configured with a sweat chemistry sensor for post-stroke rehabilitation and monitoring. In this exemplary embodiment, a wearable electronic forearm sleeve applies electrical signals causing involuntary muscle contractions in a stroke paralyzed limb.

Involuntary limb movement synchronized to audio and visual sensory cues from a VR headset invoke neuroplasticity, rewiring the stroke damaged brain. Biofeedback chemical biomarkers indicating neuroplasticity are detected using a sweat chemistry sensor.

About 66% of stroke survivors have upper limb disability. The limb is still viable, no immediate damage is done, but the “neural connection” is damaged—the stroke has impaired the part of the brain that controls the upper limb movement.

The standard-of-care is physical therapy with an attempt to re-establish the brain/body connection through physical manipulation of the stroke-affected limb. Post-stroke rehabilitation is often described as “too little, too late.” Therapy usually ends a few months after the stroke due to cost, and at-home therapy is infrequent due to limited availability of trained personnel, leaving the patient without full recovery of upper limbs. Post-stroke recovery happens when a skill that seemed lost due to the stroke is regained as the brain finds new ways to perform tasks by effectively rewiring the damaged neuronal pathways. This rewiring, known as neuroplasticity, requires an impetus that triggers this inherent capability of the central nervous system, mere repetition of movement (i.e., conventional PT) is not a sufficient impetus.

Stroke researchers have observed that challenging task-oriented training programs enhance learning and recovery. This has been demonstrated through Virtual Reality (VR) training systems, which displayed induction of brain cortical reorganization in stroke patients observed under fMRI during the training. There is a further need for measuring the invocation of neuroplasticity to monitor stroke recovery. Very little work has been done to link chemical biomarkers with the post-stroke recovery.

Accordingly, there is a clinical need for a system that will invoke and monitor neuroplasticity in real-time. Ischemic or hemorrhagic stroke is the leading cause of long-term disability. According to the World Health Organization, five million people remain permanently disabled post-stroke each year. In the U.S., more than 795,000 people suffer from stroke every year, and 25% of these people have a history of previous stroke. Over four million stroke survivors are living in the U.S. and two-thirds of them are currently disabled and might receive rehabilitation services after hospitalization. The access to a physical therapist varies depending on the geographic region, and usually insurance reimbursements are exhausted long before full recovery.

The ReHaptic device comprises a wearable electronic sleeve that applies electrical signals for muscle stimulation, causing involuntary limb movement synchronized with audio and video cues provided through a VR headset. The commercializable prototype includes a wearable electronic sleeve, a Bluetooth module for wireless communication and a wireless VR headset.

A novel sweat chemistry sensor is also integrated into the wearable electronic sleeve. This sensor first generates a small amount of sweat on-demand by local electrical and/or chemical stimulation of sweat glands under a vapor barrier. The vapor barrier coalesces sweat droplets into a liquid bio-sample. The sample is transported to a proprietary graphene field effect transistor (g-FET) biosensor. The g-FET biosensor can be configured to non-invasively identify and monitor neurotropic chemical biomarkers that are present in sweat. The potential for detecting and monitoring neurotropic biomarkers in sweat has many applications beyond stroke-rehabilitation (e.g., Alzheimer's Disease and Parkinson's Disease),

As an example, SNAP-25 is a neuronal protein biomarker that is known to be carried in blood serum. Many proteins in blood are also present in sweat and the detection of the neuronal protein biomarker is indicative of rehabilitation progress.

An exemplary embodiment uses proprietary electrical muscle stimulation wearable electronics and VR to aid in the rehabilitation of stroke patients. The system uses biometric measurement of electronic biomarkers including electromyography (EMG), as measurable signals to track rehabilitation progress and provide feedback for triggering involuntary muscle contractions through electric muscle stimulation. Other biomarkers include changes in skin temperature that are measured by the ReHaptic system to compare the temperature of the affected limb with the skin temperature of the healthy limb. The system also provides immersive sensory experiences to help rebuild lost muscle memory and neural patterns, re-educate muscle movements and fortify the brain against further damage. The simultaneous stimulation of sensory centers of the brain along with the guided limb movement caused by the involuntary muscle contractions can potentially provide the level of engagement by the brain to invoke neuroplasticity and either recruit and strengthen the still existing neuronal connections not lost due to the stroke and/or over time create new neuronal pathways that take over the motor control functions that have been lost.

As an example of a measurable biomarker indicating neuroplasticity, A novel sweat chemistry sensor is also integrated into the wearable electronic sleeve. This sensor first generates a small amount of sweat on-demand by local electrical and/or chemical stimulation of sweat glands under a vapor barrier. The vapor barrier including a proprietary structure that coalesces sweat droplets into a liquid bio-sample. The sample is transported to a proprietary graphene field effect transistor (g-FET) biosensor. Our g-FET biosensor will be configured to non-invasively identify and monitor neurotropic chemical biomarkers that are present in sweat. The potential for detecting and monitoring neurotropic biomarkers in sweat has many applications beyond stroke-rehabilitation (e.g., Alzheimer's Disease and Parkinson's Disease),

As an example, SNAP-25 is a neuronal protein biomarker that is known to be carried in blood serum. Many proteins in blood are also present in sweat, although research is needed to detect SNAP-25 in sweat.

Biofeedback is provided in the form of EMG and along with an accelerometer onboard the wearable electronic, the location of the EMG signals indicates a control intention of the patient to move the affected limb (that is, which muscles are producing the EMG signal and the signal strength help determine the movement intention as well as the degree of brain/body connection improvement). The goal is to guide the movement of the affected limb using the applied electric muscle stimulation (EMS) signals synchronized with the VR audio and visual cues. Earlier experiments by Kinaptic, have demonstrated that this guided movement creates very consistent finger and hand movements. Although precise finger control is technically challenging with electrodes only provided on the upper forearm, this low-resolution guided movement will be potentially sufficient to markedly improve the brain/body connection of the stroke survivor.

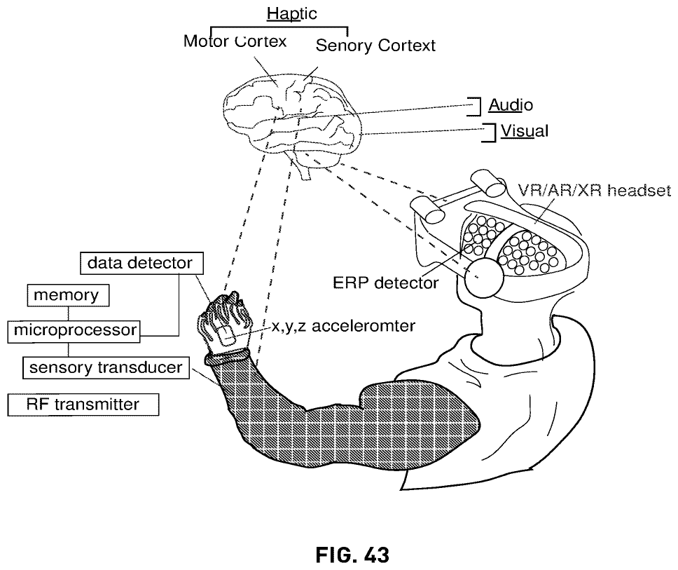

FIG. 43 illustrates an embodiment of the HHMI wearable electronic in combination with a VR headset with forward looking cameras for simultaneously applying involuntary muscle movement and haptic sensation with synchronized virtual reality audio and video sensory cues.

The Haptic Human-Machine Interface (HHMI) garment is coupled with audio and video VR cues and Electrical Muscles Stimulation (EMS), Electromyography (EMG), and Virtual Reality (VR) are used to create an at-home or in-clinic rehabilitation system for brain injury, an accelerated learning system, and a human/machine interface for use with remotely located drones, robots, deep sea or space probes, or to create a virtual experience of another time, location, being or object.

An event-related potential (ERP) is the measured brain response that is the direct result of a specific sensory, cognitive, or motor event, it can be any electrophysiological response to a stimulus. The event acquisition imager can include an ERP detector as a noninvasive measurement of brain function.

The magnetoencephalography (MEG) equivalent of ERP is the ERF, or event-related fields. Evoked potentials and induced potentials are subtypes of ERPs. Electromyoprahy is the detection of electric potential generated by muscle cells when these cells are electrically or neurologically activated. The signals can be analyzed to detect abnormalities, activation level, or recruitment order, or to analyze the biomechanics of human or animal movement. The HHMI described herein can utilize ERP, EEG, MEG, EMS, EMG, and other biometric measurements and applied electrical and haptic signals to create a wearable electronic system for a wide variety of use-cases.

Neurorehabilitation is a successful method to induce optimal motor recovery post-stroke. Neurorehabilitation relies on a series of interventions based on human and animal studies regarding learning and adaptation, and the activation of experience-dependent neuronal plasticity in augmenting functional recovery after stroke. There are several modes of intervention that have been observed to be successful. The HHMI can be configured to provide these modes of intervention in an easy to use, easy to wear, low cost rehabilitation system that can be used in a clinical setting or at-home. Stroke often leads to neuronal death and damage to the motor cortex leading to decreased motor capabilities. The brain can form new connections to compensate for a part of the loss, which is enhanced in the critical period of 1-3 months after the stroke. Neuroplastic changes in the cerebral cortex are linked to motor skill retraining in the affected limbs. Task-oriented training focuses on the practice of skilled motor performance important for activities of daily life (for example drinking a glass of water, answering the telephone, etc.) that facilitates neural reorganization in the brain. Hence, task-oriented training has been the newly accepted approach to stroke rehabilitation. In particular, Repetitive task training, which recommends redoing the task-oriented tasks multiple times during the day has been shown to produce functional recovery in stroke patients. People who received repetitive task training showed greater improvements in performing functional tasks, such as picking up a cup, standing up, and walking. These improvements were sustained for up to six months. Particularly for the upper limb, it showed improvement in multiple measures such as Action Research Arm Test, Time to complete Jebson Taylor Hand Test, Sitting Equilibrium Index, Lateral Reach Test, Maximum reach distance, Motor Assessment Scale—upper arm, Functional test of the hemiparetic upper extremity and Wolf Motor Function Test.

Evidence from animal studies also suggests that greater reductions in poststroke motor impairment can be attained with significantly higher doses and intensities of therapy focused on movement quality. These studies also indicate a dose-timing interaction, with more pronounced effects if high-intensity therapy is delivered in the acute/subacute, rather than chronic, poststroke period. Neuro-animation may be an efficient way to provide high-dose and intensive upper-limb therapy in stroke rehabilitation compared to the traditional approach of low-intensity interval therapy. Such an intervention showed significantly greater recovery on Action Research Arm Test (ARAT) scale.

Resting-state functional connectivity of the primary motor cortex was observed in stroke patients before and immediately after 4 weeks of robot-assisted bilateral arm therapy (in which the patient holds a robotic arm each in the left and right hands and moves it around during therapy). A significant improvement was reported post-treatment on all clinical measures including Fugl-Meyer Assessment (FMA) score, Wolf Motor Function Test (WMFT) score, and Functional Independence Measure (FIM). The HHMI uses the body's own mechanical and electrical system to provide therapy that is similar to the robot-assisted therapy.

EMG, combined with Comprehensive Rehabilitation Training, can effectively improve multiple motor functions including limb movement, balance, daily life ability. FMA, M-B index (MBI), Berg Balance Scale (BBS), score before and 4 weeks after the treatment of the EMG biofeedback treatment group were observed to be significantly better. The HHMI monitors the body's own mechanical (accelerometer) and electrical (EMG) biometrics.

In summary, multisensory feedback therapy combined with task-oriented training is considered to be advantageous and effective in stroke rehabilitation. The HHMI combines virtual reality (VR), Electromyography (EMG), and Electrical Muscle stimulation (EMS) to allow increased chances of restoration of functions of the stroke-impaired upper limbs after the critical care period (2-3 months) has ended. The solution uses an electrical muscle stimulation generated by a patented wearable electronic garment for the upper limb to create involuntary muscle contractions which are synchronized with audio and video sensory cues provided by a virtual reality headset. The objective is to promote functional recovery based on the evidence that both virtual reality as well as biofeedback can enhance brain plasticity which is essential for recovery. Combining the detection of EMG with EEG, directly measures the progress of a patient towards functional recovery. Artificial intelligence paradigms can be applied to unlock the utility of multiparametric data analysis of continuous EEG, EMG, and movement kinematics from an accelerometer during the acute phases of stroke care. The goal is to obtain a determinative biomarker from EEG in combination with accelerometer and EMG data from HHMI to determine rehabilitation effectiveness and modifications needed for rehabilitation optimization.

As shown in the embodiments described herein, EMS stimulation is synchronized with the VR audio and video feed which ensures an immersive experience for an effective and stimulating rehabilitation program. Variations in the EMG activity of specific muscles quantifies the patient's recovery and is used as biofeedback to automatically select the best rehabilitation protocol. An embodiment disclosed herein can be used for upper limb recovery and also adaptable for lower limb applications. An advantage of the HHMI rehabilitation system is the continuous adaptation of rehabilitation exercises and stimuli based on patient biofeedback recovery data. The HHMI is useful as a neurorehabilitation device providing wearable device that is driven by biometric recovery data. The naturally occurring electrical systems of the human body are used to overcome and mitigate the dysfunction and challenges caused by a stroke damaged brain and the brain's inherent neuroplasticity is invoked to rewire the damaged brain and restore a high degree of functional life skills and cognitive capability back to the stroke victim. The HHMI opens new avenues in human/machine interaction and control, that also impact areas of accelerated learning, physical training, entertainment, remote drone control.

Stroke causes a dramatic alteration of neural networks within the brain's affected area. It has been amply demonstrated using functional MRI (fMRI) that the cerebral cortex exhibits spontaneous brain plasticity response to damage which causes a reorganization of the neural connection and this rewiring is highly sensitive to the experience following the damage. The HHMI provides a post-stroke rehabilitation device based on Electrical Muscles Stimulation (EMS), Electromyography (EMG), and Virtual Reality (VR). EMS-based involuntary stimulation of impaired muscles is achieved through a wearable electronic garment and is synchronized with the VR audio and video feed to provide an immersive experience for an effective and stimulating rehabilitation program. Variations in the EMG activity of specific muscles quantifies the patient's recovery and it is used as biofeedback to automatically select the best rehabilitation protocol.

The use of involuntary muscle contractions synchronized with visual and audio VR cues will enable immersive exercises, effectively invoking neuroplasticity and activating brain rewiring. This feature will guarantee a faster and more effective long-term recovery from mobility impairments. The HHMI can also provide biometric data by measuring the electrical EMG signal that quantifies muscles electrical activities. This data quantification of the recovery speed, to personalize the rehabilitation process and enhance patient rehabilitation.

The HHMI uses VR, EMS and EMG to realize a closed-loop biofeedback system to deliver superior rehabilitation and monitor at the same time patient's recovery to increase engagement and activate brain rewiring. A non-limiting embodiment includes a configuration of the HHMI for upper limb rehabilitation with a stretch fabric wearable electronic sleeve that is thin, lightweight, and comfortable. It is designed to be worn under clothing, with self-contained thin and flex-circuit based electronics. The HHMI stimulates the patient with Electric Muscle Stimulations (EMS) and Transcutaneous Electrical Nerve Stimulation (TENS) to induce involuntary muscle contraction, provide haptic stimulus and acquire biofeedback signals including Electromyography (EMG) and accelerometry. A Video/Audio VR engine provides inputs synchronized with the HHMI to stimulate the Occipital lobe (visual cues) and Temporal lobe (audio cues) for a deep VR immersion combined with brain rewiring stimulation. Virtual Reality scenes are synchronized with a minimal time delay. Use of the HHMI creates brain rewiring through biofeedback acquisition and VR immersivenes and exploits haptic, audio & video VR to activate brain neuroplasticity.

The HHMI adapts the rehabilitation plan for a patient based on the patient's own biometric data, including EMG biofeedback in response to an applied treatment. This feature enables the obtainment of quantitative information on patient progression used to optimize the rehabilitation process while minimizing physician involvement.

Due to the slow re-growth rate of dead neuronal cells in damaged regions, cellular recovery is unlikely. Nevertheless, it has been demonstrated that, following ischemic injury, the brain is capable of considerable reorganization or “rewiring”, and this phenomenon can form the basis for functional recovery. In particular, depending on the topography of the injury, the location of cortical function can be displaced to neighboring areas. This relearning process is mediated by neuroplasticity, which is defined as the ability of the central nervous system to undergo structural and functional change in response to new experiences. Neuroplasticity is consistent with motor learning in adults who are healthy, leading to the learning of new tasks. Neuroplasticity has been widely confirmed in humans with a number of experimental techniques, including noninvasive brain stimulation and functional magnetic resonance imaging (fMRI). Learning-related plasticity involves the formation of new neural connections that support learned behaviors followed by pruning, or “focusing,” of neural connections as skill and preferential pathways develop.

Stroke rehabilitation focuses on maximizing functional recovery by enhancing physiological motor learning-related neuroplasticity. Patient engagement is critical for successful stroke rehabilitation. Importantly there are demonstrated relationships among dendritic growth, structured environmental stimulation, and the recovery of lost functions. Studies examining the links between training, motor learning, neuroplasticity, and improvements in hand motor function showed that neuroplasticity can be effectively instigated for stroke rehabilitation utilizing simulated activities that focused on the recovery of the damaged body area. Recent academic research has identified patient engagement and motivation to significantly improve rehabilitation outcomes. Patient tailored rehabilitation is key of success. The HHMI collects biofeedback from patients (EMG, movement data) and uses this data to adjust the rehab protocol on a personalized basis. The level of immersion in the environment plays an essential role in providing an optimal condition for task practice as does the meaningfulness of the task to the participant. In particular, high repetition intensity, salient task practice, novel environments offering high motivation and enhanced feedback (visual, auditory, haptic) on the results of performance are critical features for rehabilitation success provided by the use of the HHMI. The HHMI used for VR stroke rehabilitation represents the best framework for stroke rehabilitation as environments can be easily manipulated to fulfill all key training criteria, engaging the patient in a creative, realistic and demanding effort.

The HHMI simultaneously stimulates several portions of the brain related to the processing of sound, touch and vision so that a weakened brain's processing center can be strengthened or rewired through the support of stronger, intact, brain sensory stimulation processing centers. These touch and movement sensory cues stimulate the damaged portions of the brain, while corresponding, synchronized virtual reality visual, audio, and haptic cues reinforce the re-learning or rewiring of the damaged portions of the brain.

The HHMI applies electrical signals for Electric Muscle Stimulation (EMS) to provide haptic feedback and involuntary movement, and to acquire Electromyography (EMG) signals. A conductive fabric in contact with the skin applies the EMS neuromuscular stimulation for involuntary muscle contraction, for example, as a low frequency (<100 Hz) pulse which triggers the alpha motor nerves controlling muscle movement. The signal is applied to each muscle or group of muscles, by two or more individually addressable electrodes. The resulting contraction depends on the applied signal: the intensity (e.g., 0-100 mA) controls the amount of muscle fibers excited and thus the contraction strength, whereas the signal shape (polar/bipolar) controls the type of muscle contraction (isometric/isotonic), the contraction speed, and the duration. The TENS signal for haptic feedback selectively stimulates several receptors with different receptive fields (typical range: 20-80 mA and 0.4-800 Hz). The EMG signal is the electric signal that the brains sends to the muscle to trigger the movement. This signal is typically in the range [30-100] mA and [0-100] Hz for healthy individuals, and can be reduced down to [0-25] mA and [0-100] Hz in stroke patients. This signal is read as the electric current generated by potential difference between the muscle and the reference electrode.

The HHMI VR stroke rehabilitation system is well suited to provide skill training with immediate and accurate performance feedback through visual, auditory, and haptic rewards, increasing a patient's motivation to practice the virtual tasks that lead to regaining real-world life skills. Binaural audio (i.e. audio recorded in 3D) can be utilized to deepen immersion and realism.

The HHMI acquires biofeedback data from EMG sensors and from accelerometers located on the HHMI. This data gives the patient and the physicians access to information about physiological functioning. Accelerometry and EMG data can be extracted continuously, after each rehabilitation exercise, etc., and analyzed to assess if the patient's movement and brain response are improving over time and if they reached recovery thresholds.

In an experiment to determine adequate applied EMS signal intensity to cause involuntary movement without discomfort, the HHMI was worn by a healthy volunteer with his hand and wrist relaxed, and EMS signals with increasing intensities (0-75 mA at 50 Hz) were applied. The sensation intensity (i.e. the ability of volunteer to feel the applied EMS signal, on a scale 1-10) and the discomfort level (the level of comfort/discomfort caused by the EMS sensation, on a scale 1-10) reported by the volunteer, together with the observed hand movement were recorded. Results: If no EMS signal or low intensity (<25 mA) EMS signals are applied, the hand drops due to gravity to a resting position. For intermediate EMS intensities [25-70] mA, a partial involuntary hand movement is observed, indicating that a threshold to cause the hand to pivot at the wrist is reached. For high EMS intensities (>70 mA), the hand reaches its maximum rotation point around the wrist (i.e. equal to the maximum degree of stretch the test subject can achieve voluntarily) indicating that involuntary muscle contraction is complete. The volunteer reported slight sensation from the applied EMS signal starting at 7 mA with its magnitude constantly increasing with the applied EMS signal intensity Although the volunteer reported an increase in sensation, no hand movement was observed until an EMS signal of 25 mA was applied. The volunteer reported that the applied EMS signal was comfortable and a pleasant sensation up to 90 mA. At no point did the test subject indicate that the applied EMS signal was intolerable, but in the [90-100] mA range, the volunteer reported the EMS signal was no longer a pleasant sensation. Thus, an EMS signal in the 25-90 mA range can induce complete involuntary movement without inducing discomfort.

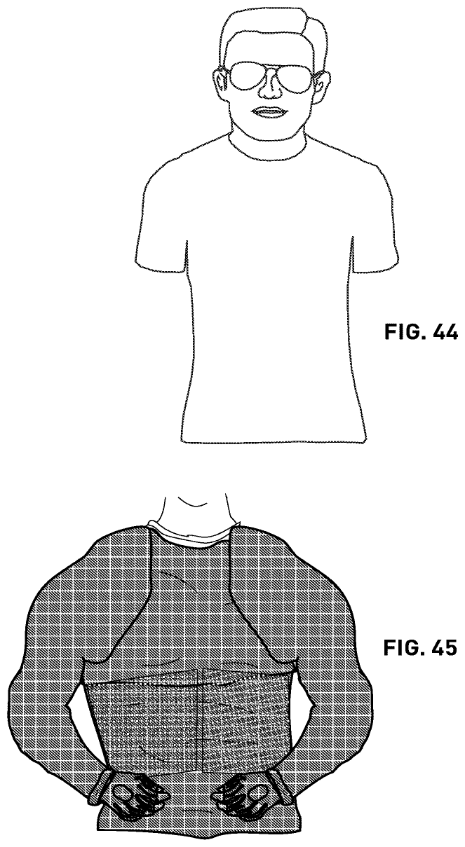

FIG. 44 illustrates an HHMI pixelated shirt in combination with forward looking cameras and VR/AR/XR head gear providing a wearable electronic for haptic vision.

The HHMI can be configured as a wearable electronic for providing haptic vision, where the large surface area of the skin of the user is made available as an information source for the user's brain to discern stationary and moving objects ahead of or surrounding the user. FIG. 45 illustrates an HHMI pixelated shirt in combination with forward looking cameras and possibly VR/AR/XR head gear. The HHMI pixelated shirt can be used for a haptic vision use-case.

FIG. 45 illustrates the HHMI pixelated shirt for a haptic vision use-case.

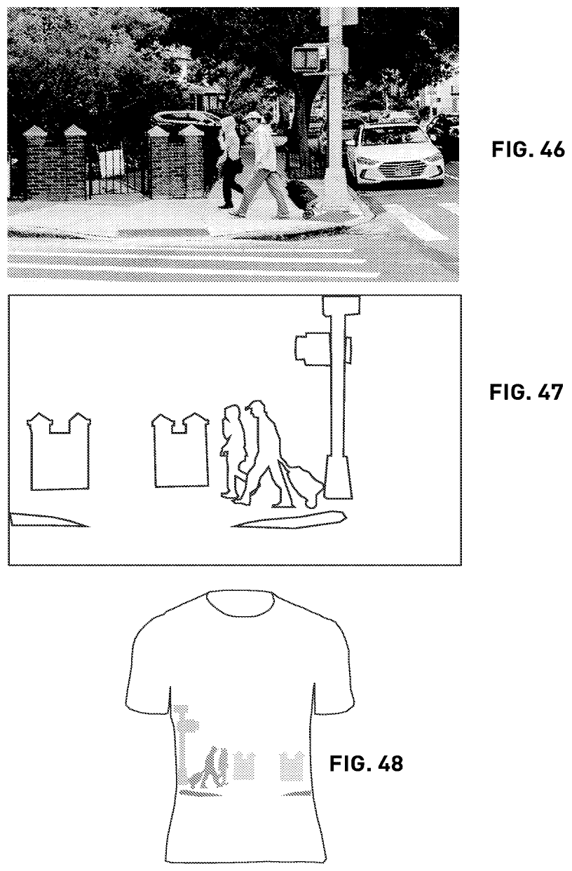

FIG. 46 shows a scene as a visually impaired user wearing the HHMI pixelated shirt approaches a cross walk.

The haptic vision HHMI comprises an event acquisition imager for acquiring at least one image to display to a wearer; a skin-contact information display system having individually addressable pixels for providing information to the wearer through at least one of haptic sensations and involuntary muscle contractions. A processor determines an electrical signal applied to the individually addressable pixels to convey the image to the user through the information provided by the at least one of haptic sensations and involuntary muscle contractions.

FIG. 47 shows the scene of the cross walk reproduced as haptic sensations on skin of the torso of the visually impaired user.

FIG. 48 shows a smart shirt having a skin-contact information display system comprising individually addressable electrodes for applying electrical signals and/or haptic sensations, such as vibrations using localized and addressable vibrators.

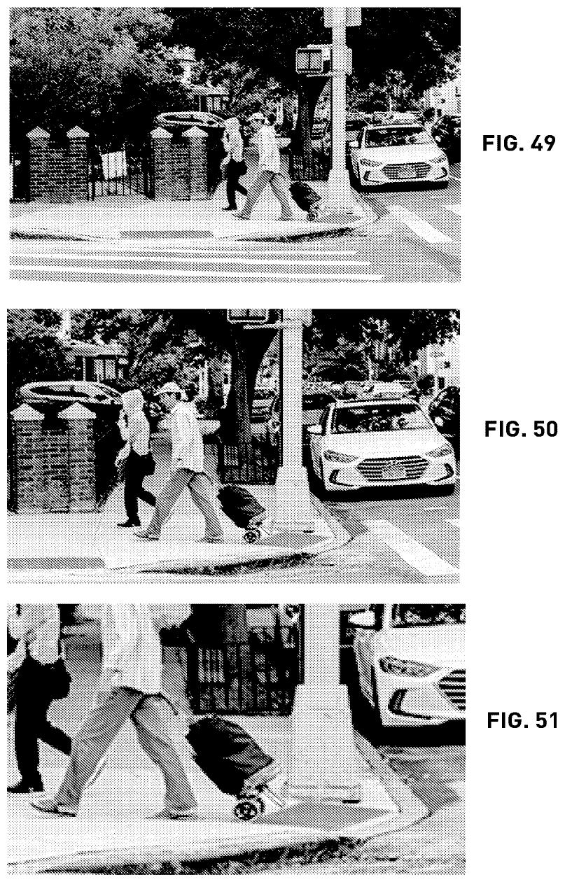

FIG. 49 shows the scene of the cross walk at the farther distance from the visually impaired user walking towards the other side of the street.

FIG. 50 shows the scene of the cross walk as the visually impaired user comes closer to the other side of the street.

FIG. 51 shows the scene of the cross walk as the visually impaired user comes even closer to the other side of the street.

FIG. 52 illustrates how the scene of the cross walk is reproduced on there HHMI pixelated shirt as some scene elements remain stationary and some are in motion.

FIG. 53 illustrates how the scene reproduced on the HHMI pixelated shirt indicates to the visually impaired user where elements of the scene are in motion and others remain stationary.

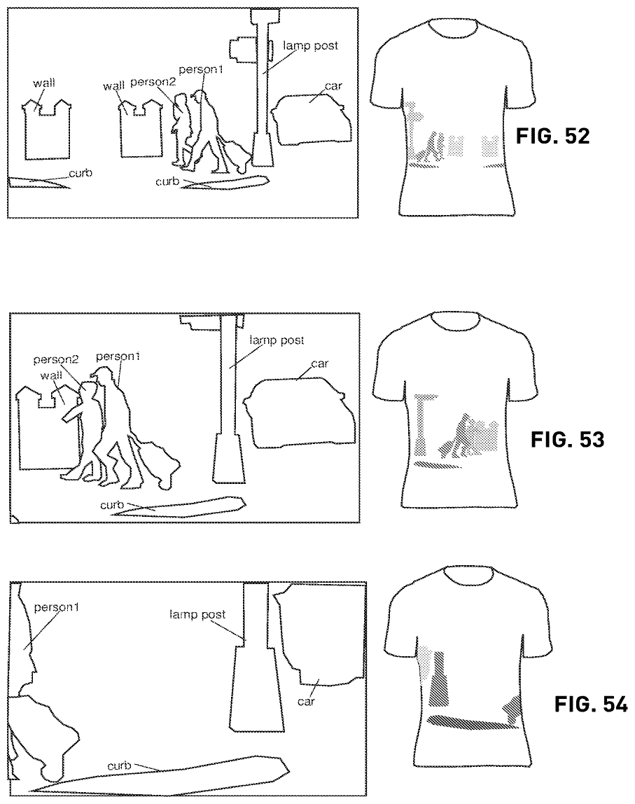

FIG. 54 illustrates how elements of the scene are proportionally increased in size and haptic signal intensity as the visually impaired user approaches the other side of the street.

In accordance with a non-limiting embodiment, the individually addressable pixels comprise skin contact electrodes. The event acquisition system can include at least one of a camera, radar, lidar, thermal imagining, UV imagining, IR imagining, VR system, AR system, and an XR system. The acquired image can be dependent on the ambient surroundings of the wearer, and/or dependent on the ambient surroundings of an object remotely located from the wearer. The remote object can be at least one of a, drone, different human, or animal. The VR scene can provide sensory cues of a least one sense, including vision, hearing, smell, taste, feel, to the user so that involuntary muscle contractions and haptic sensations are applied through the skin of the user synchronously with the VR scene.

The wearable electronic shirt helps visually impaired users to experience visual information through their skin. The shirt has pixelated electrodes and/or vibrators that can be individually actuated, creating a haptic “image” of a scene.

The technology comprises an event acquisition system that includes at least one type of sensor, such as a camera, radar, lidar, thermal imagining, UV imagining, IR imagining, VR system, AR system, or XR system. This system acquires an image that can be dependent on the ambient surroundings of the wearer, and/or dependent on the ambient surroundings of an object remotely located from the wearer. The remote object can be a drone, a different human, or an animal.

The wearable electronic shirt can also be used by any user and integrated with a VR system that provides sensory cues of at least one sense, including vision, hearing, smell, taste, and feel, to the user. Involuntary muscle contractions and haptic sensations are applied through the skin of the user synchronously with the VR scene. This provides a fully immersive experience for the user, enabling them to not only visualize the scene but also feel it through their skin.

The individually addressable pixels of the wearable electronic shirt comprise skin contact electrodes that can be actuated to replicate the visual image obtained by the forward-looking cameras worn by the user. The image is translated into a haptic image, which is sent to the user through the electrodes in the shirt. This creates a sensation that mimics the visual image, enabling the user to “see” the image through their skin.

The technology has the potential to revolutionize the way visually impaired individuals experience the world around them. It allows them to receive visual information in a tactile manner, enabling them to navigate their surroundings more effectively. Additionally, the integration of VR technology with the wearable electronic shirt provides a fully immersive experience, enabling visually impaired users to experience a world that was previously out of reach.

The wearable electronic shirt has the potential to change the lives of millions of visually impaired individuals worldwide. It combines cutting-edge sensor technology with advanced haptic feedback to create an experience that is truly unique and transformative. The ability to “see” through one's skin has the potential to open up a world of possibilities for visually impaired individuals.

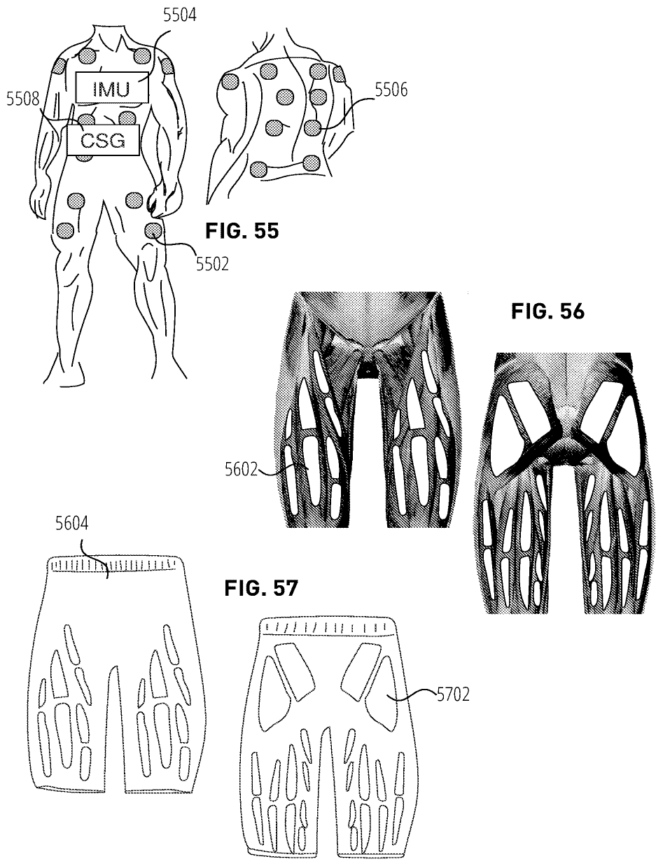

FIG. 55 shows locations of skin contact electrodes and position detectors for an embodiment of the wearable electronic garment used for gait disorders. The system comprises multiple electrodes 5502, an Inertial Measurement Unit IMU 5504, the Balance Control Muscles 5506 of the user, and a Core Stabilizing Gyroscope 5508.

In one embodiment, the present invention relates to a non-pharmacological, non-surgical, electroceutical therapeutic configured as a wearable electronic garment for the treatment of movement disorders. The wearable electronic garment utilizes electromyography, accelerations and inertia changes of the body to detect and analyze movement and determine the electrical characteristics of haptic sensations and EMS signals to be applied to the body. The EMS signals are applied to the Balance Control Muscles (BCMs) through transcutaneous electrical stimulation, causing computer-controlled sensory perceptions and involuntary muscle contractions to mitigate the movement disorder.

In this aspect of the invention, the wearable electronic garment detects Movement Disorder Motion (MDM) through accelerations and inertia changes of the subject wearing the garment, and electromyography detection of the actual MDM-involved muscles. For example, an IMU can be used as a sensor located on the patient or user's body. An IMU 5504 is an electronic device that measures and reports the orientation, velocity, and acceleration of an object, typically with respect to an inertial reference frame. It consists of a combination of sensors, such as accelerometers, gyroscopes, and magnetometers, which work together to provide a complete picture of an object's movement and orientation in three-dimensional space. IMUs are commonly used in robotics, aerospace, navigation systems, and motion capture technology. In accordance with this aspect of the invention, an IMU can also be used in wearable electronic devices to detect movement and changes in orientation of the body.

The detected information is analyzed to determine MDM-opposing muscles and MDM mitigation signals, which are applied to stimulate the MDM-opposing muscles. The wearable electronic garment targets the muscle/nerve motor units resulting in MDM, with the mechanism of action for mitigation being MDM-opposing muscle contractions that restrain the MDM.

As described in U.S. Pat. No. 0,437,335, issued Oct. 8, 2019 to John James Daniels, the disclosure of which is incorporated herein in its entirety, movement disorders are chronic, often painful and debilitating conditions that affect the ability to control movement. Having a movement disorder can make it difficult, even impossible, to do the routine things in life. More than 40 million Americans—nearly one in seven people—are affected by a movement disorder, including tremor, Parkinson's disease, Tourette's syndrome, dystonia, Multiple Sclerosis, and spasticity. The wearable electronic garment can be for treatment of balance and gait disorders. Vision, the vestibular system and the somatosensory system all work harmoniously to maintain posture and balance in a healthy individual. To further enhance the effect of the applied electroceutical treatment, the wearable electronic can also be used in combination with a core-steadying gyroscope.

The wearable electronic can be configured with a core-stabilizing gyroscope adjacent to the chest of the wearer. A review of the reference literature indicates that the swaying of a healthy individual while maintaining balance can be modeled as an inverted pendulum. The wearable electronic can use a detectable EMG signal that corresponds to the muscle groups that are activated to maintain the inverted pendulum sway (i.e., the BCMs or Balance Control Muscles 5506), and that up to a point (determined by the IMU or the spinning gyro-scope mass and rotational speed), the swaying of the torso core will undergo proportional inertial resistance from the gyroscope. The EMG, movement and inertia data can be detected at the limbs, BCMs and torso core, etc. The BCMs are determined and involuntarily activated and if present, in conjunction with the core-steadying gyroscope. The wearable electronic is applicable, among other uses, to cognitive therapy, accelerated learning, brain/spinal cord rehabilitation, balance restoration and tremor mitigation.

Electromyography detects the actual MDM-causing muscles. The detected information is analyzed to determine MDM-opposing muscles and MDM mitigation signals. The MDM mitigation signals are applied to stimulate the MDM-opposing muscles. The target for detection is the muscle/nerve motor units resulting in MDM, with the mechanism of action for mitigation being MDM-opposing muscle contractions that restrain the MDM.

Vision is primarily used in movement planning and avoiding obstacles. The vestibular system senses linear and angular accelerations, and the many sensors of the somatosensory system are used to determine body part position, contact and orientation

FIG. 56 shows some of the upper leg muscles that can be used as Balance Control Muscles. Balance Control Muscles 5602 are in contact with electrodes provided on a wearable electronic garment 5604, and an applied sequence of EMS signal to these BCMs return or guide the patient's body to an upright position when a potential loss of balance is detected.

In accordance with an embodiment, an apparatus for mitigating gait disorders is provided where the apparatus stores initial baseline biometric data in a memory, where the initial baseline biometric data is obtained using a baseline biometric test. The baseline biometric can be at least one of EMG signals from Balance Control Muscles that are activated when the user is standing upright and trying to maintain balance and/or when presented with a force such as a push or an inclined treadmill. The baseline biometrics can alternatively or in addition be accelerometer or IMU data from the user trying to maintain balance. This baseline biometric can be used later to indicate the signal characteristics and location of application on the user's body of haptic sensations and EMS signals applied in response to monitored biometric parameters including EMG, accelerometer and IMU data indicating a problem with the user's gait or balance.

At least one patient-specific threshold is determined for one or more monitored biometric parameters dependent on the stored baseline biometric data. For example, the location of EMG signals that the user's body creates when trying to maintain balance can be used to indicate which muscles to apply an EMS signal to assist the user in maintaining balance. The accelerometer and/or IMU data can be used to indicate when the user is beginning to lose their balance.

During use of the wearable electronic for mitigating gait disorder, one or more monitored biometric parameters are detected using biometric sensors. The sensors may have the same, or different relative accuracy than sensors used to obtain the baseline biometric. When at least one threshold is exceeded, such as an acceleration of a body part, e.g. the torso, towards a tipping point at a speed that indicates the patient may be in danger of losing balance, EMS signals are applied to the appropriate Balance Control Muscles to help the patient maintain balance. The strength, duration, sequence and number of applied EMS signals to the Balance Control Muscles can be adapted to each individual patient depending on their specific requirements for maintaining balance. For example, in some instances and for some patients, they may need just a gentle indication through haptic and/or applied EMS signals that there is a danger of losing balance. In other instances, and for other patients, a stronger applied EMS signal may be needed to cause involuntary muscle contractions that straighten their body up and prevent falling over.

The wearable electronic garment addresses gait disorders by detecting when a patient is losing balance and using Body-in-the-Loop™ feedback to apply electrical muscle stimulation to the Balance Control Muscles (BCMs) of the lower legs and torso to guide the body back into balance. The wearable electronic garment provides a non-invasive, adaptable, and targeted intervention that can be used alongside existing treatments to improve patients' mobility, stability, and overall quality of life.

The present invention has particular utility in the treatment of gait and balance disorders in patients with neurological conditions, including multiple sclerosis, Parkinson's Disease, etc. As an example, MS affects approximately 2.8 million people worldwide and often results in gait and balance disorders, posing significant challenges in patients' daily activities and increasing the risk of falls and injuries. The wearable electronic garment offers personalized, real-time assistance for patients experiencing gait and balance disorders, reducing fall-related injuries, minimizing reliance on assistive devices, and enhancing the overall management of gait and balance disorders in MS and other neurological conditions.

In accordance with another aspect of the invention, a non-pharmacological, non-surgical, electroceutical therapeutic for the treatment of movement disorders is configured as a wearable electronic garment. Electromyography, accelerations and inertia changes of the body are sensed to detect movement. This detected movement is analyzed to determine electrical characteristics of haptic sensations and EMS signals to be applied to the body. EMS signals are applied to Balance Control Muscles (BCM) through transcutaneous electrical stimulation causing computer-controlled sensory perceptions and involuntary muscle contractions. The applied haptic and EMS signal mitigate the outward and inward symptoms of movement disorders. Accelerations and inertia changes of the user wearable the wearable electronic garment for detecting and treating Movement Disorder Motions (MDMs). Electromyography detects the actual MDM-involved muscles. The detected information is analyzed to determine MDM-opposing muscles and MDM mitigation signals. The MDM mitigation signals are applied to stimulate the MDM-opposing muscles. The target for detection is the muscle/nerve motor units resulting in MDM, with the mechanism of action for mitigation being MDM-opposing muscle contractions that restrain the MDM.

FIG. 57 shows a pair of skin fitting shorts with skin contact electrodes for applying EMS signals to BCMs of the upper legs. The shorts include electrodes 5702 that can apply EMS and detect EMG signals from the BCMs. Similarly, a torso suit or other garment can be configured for applying and detecting electrical signals to and from other BCMs, for example in the back, stomach and shoulders.

Gait disorders are a major cause of functional impairment and morbidity in older adults and for younger people with neurological conditions. Most gait disorders in this population are multifactorial and have both neurologic and non-neurologic components. The control of gait and posture is multifactorial, and a defect at any level of control can result in a gait disorder.

There are eight basic pathological gaits that can be attributed to neurological conditions: hemiplegic, spastic diplegic, neuropathic, myopathic, Parkinsonian, chloroform, ataxic (cerebellar) and sensory. Gait disorders contribute to reduced mobility, fall risk, diminished quality of life, and serious injuries including major fractures and head trauma. It is estimated that approximately 15 percent of falls in older adults can be attributed to balance or gait disorders, including leg weakness.

Gait and balance disorders stem from various neurological conditions, including Parkinson's disease, multiple sclerosis, stroke, cerebellar ataxia, normal pressure hydrocephalus, peripheral neuropathy, brain tumors, spinal cord injuries, myopathies, and other neurodegenerative disorders. In accordance with a non-limiting embodiment, a wearable electronic garment detects when a patient is losing balance and, using body-in-the-loop™ feedback, applies electrical muscle stimulation to Balance Control Muscles (BCMs) of the lower legs and torso to guide the body back into balance.

As an example use-case, Multiple Sclerosis (MS) is a chronic neurological disorder affecting approximately 2.8 million people worldwide, with substantial impact on patients' quality of life. MS, along with other neurological and non-neurological conditions, often results in gait and balance disorders, posing significant challenges in patients' daily activities and increasing the risk of falls and injuries.

MS is typically diagnosed between the ages of 20 and 50, but it can also occur in younger and older individuals. Women are more likely to be diagnosed with MS than men, with a ratio of approximately 2-3:1. MS is more common in Caucasians, particularly those of Northern European descent, compared to other racial and ethnic groups. African Americans, Hispanics, and Asians have a lower prevalence of MS, although recent studies have shown that African Americans might have a higher risk of developing a more aggressive form of the disease.

The prevalence of MS is generally higher in regions farther from the equator, suggesting that environmental factors, such as sunlight exposure and vitamin D levels, may play a role in the development of the disease. In the United States, it is estimated that around 1 million people are living with MS.

The standard-of-care for gait and balance disorders in MS patients involves a combination of pharmacological interventions, physical therapy, and assistive devices. Medications, such as immunomodulatory drugs, are used to manage the underlying disease progression, while symptomatic treatments and physical therapy aim to improve mobility and balance. Assistive devices, including canes, walkers, and orthotic braces, provide additional support and stability.

However, the current standard-of-care has limitations, including inadequate responsiveness to sudden balance disturbances, potential side effects of medications, and the cumbersome nature of such assistive devices. Moreover, existing solutions may not fully address the unique needs of each patient, as the severity and presentation of gait and balance disorders can vary widely.

In accordance with this aspect of the invention, a wearable electronic garment addresses these limitations and provides personalized, real-time assistance for patients experiencing gait and balance disorders. The garment can provide biometric feedback on the patient's progression of the disease to a healthcare provider, and also provide empirical data for insurance companies, NGOs and government agencies to indicate the real dollar value and monetary savings of the improved patient outcomes as a result of using the wearable electronic.

The garment detects when a patient is losing balance and employs Body-in-the-Loop™ feedback to apply electrical muscle stimulation to the Balance Control Muscles (BCMs) of the lower legs and torso, guiding the body back into balance.

This technology offers a non-invasive, adaptable, and targeted intervention that can be used alongside existing treatments to improve patients' mobility, stability, and overall quality of life. By integrating seamlessly into patients' daily routines, the wearable garment has the potential to reduce fall-related injuries, minimize reliance on assistive devices, and enhance the overall management of gait and balance disorders in MS and other neurological conditions.

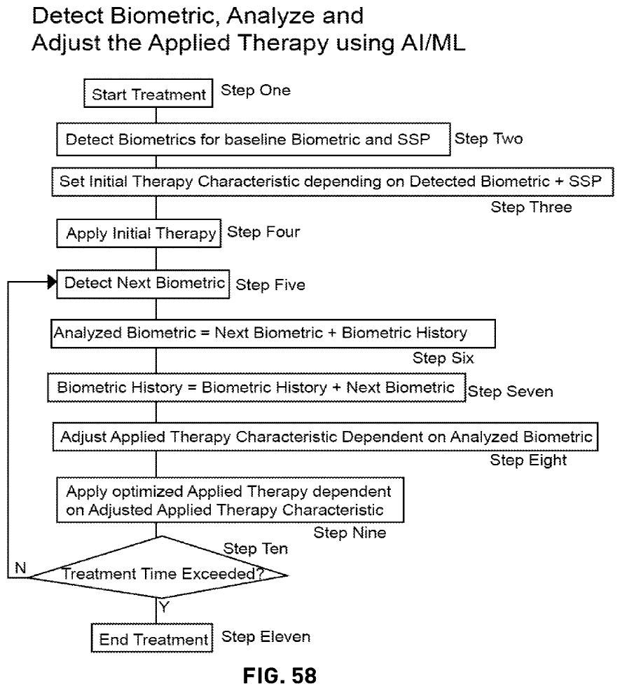

FIG. 58 is a flowchart for AI/ML adjusted therapy. Referring the flowchart of FIG. 58, the treatment is started, which may include the previous or concurrent ingestion of a pill or a transcutaneous injection of a drug, an applied electrical signal (e.g., EMS signals as described herein), the detection of a biometrics (e.g., IMU data), or the like (Step One). At least one biometric parameter is detected as a baseline, which may be detected concurrently at the start of treatment or previous to the treatment, for example to get a baseline for the patient at a doctor's office. A Similarly Situated Patient (SSP) cohort that the patient belongs to is also determined. The SSP cohort can be determined through a doctor's visit where better accuracy diagnosis and biometric determinations can be performed (as described elsewhere herein). The detected biometric and determination of SSP cohort is used to build a biometric history (Step Two). This biometric history is used to set initial therapy characteristics. For example, in the case of a gait disorder patient, the initial therapy characteristic could be the degree of restraint of movement (e.g., measured metric determined at the doctor's office) caused by the applied EMS signals (frequency, amplitude, duration, etc.) when the electroceutical therapy is used to help with balance. Typically, the degree of restraint of movement should tend towards less restraint (e.g., at an early stage of MS) and then progress towards more restraint (e.g., later MS stages). The detected biometric or biometric parameter can be, for example, EMG signals, IMU or accelerometer data, or even cellphone IMU data. In the case of cellphone IMU data, several years' worth of IMU data of the cellphone held by the patient while walking may be available to help diagnose aspects of the disease progression (onset and rate of change) and help set the initial therapy characteristics. The initial therapy characteristics are set depending on at least one detected biometric (Step Three). In the case of the MS patient, for example, the therapy may be sequentially applied EMS signals that causes BCM muscles to contract and cause the patient to stand straight up and avoid a fall caused by loss of balance.

The initial therapy is applied having the characteristic set depending on the detected biometric (Step Four). A next biometric is then detected (Step Five). The next biometric may be the same type (e.g., EMG) or a different type (e.g., body position, angle of body center of gravity relative to the ground, etc.). Each next biometric plus the biometric history (which will typically depend on previously detected biometrics) is analyzed using AI/ML to create an analyzed biometric (Step Six). The biometric history is then updated to include the next biometric (Step Seven). As a non-limiting example, the biometric can be determined using a EMG signals from the BCMs along with body position data to determine the extent of intervention necessary to avoid a fall. When successive biometrics (that is, the next biometric and the biometric history) are compared, the analyzed biometric may indicate that over time the patient is requiring more and more applied electroceutical intervention to prevent falling. In this example, it may be advantageous to adjust the applied therapy characteristic such as the thresholds that determine how much restraint should be applied to the patient's freedom of movement. For example, to increase the strength of the sequentially applied EMS signal or the duration of the sequentially applied EMS therapy (i.e., extend the treatment time duration of the applied EMS signals, so that patient is more quickly brought to an upright position and held there through the applied EMS for a longer duration to regain balance). In this way, the adjusted applied therapy characteristic is used to optimize the applied therapy that is applied to the patient (Step Nine). If the treatment time is exceeded (Step Ten) the treatment is ended (Step Eleven), otherwise the next biometric is detected (Step Five) and the flowchart steps continue. The treatment time may be exceeded, for example, the patient has returned to a stable upright position, in which case, the next time the IMU or body position data indicates a loss of balance, the Start Treatment (Step One) can begin again.

If the patient continues to tip and lose balance, that tipping can be determined through the detected next biometric and the patient's body become part of a feedback loop where the balance is restored through involuntary muscle contractions and possibly the brain learns how to cope with a loss of balance or gait functionality. Over time, this algorithm will build up a history of relevant patient-generated biometric data that can be used for Big Data analysis to improve the wearable electronic as a gait disorder treatment for the SSP cohort, other use-cases and also optimized for the individual patient with adjustments automatically made as the disease progresses.

In addition, the feedback information obtained using the gait disorder wearable electronic garment can be provided to the collection of Big Data for the SSP cohort, so that this learned information and AL/ML analysis becomes part of the improvement of the system that is made available to other users of the wearable electronic.

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

The embodiments described herein are intended to exemplary and non-limiting, the selection of biometric, environmental, or other measured conditions is not limited to a specific metric or multiple metrics described herein but will depend on the particular application and treatment, data collection, and/or other use of the detected metrics. Also, the treatments employed in any of the embodiments described herein is not limited to a specific treatment or action but will depend on the intended use and desired outcome of the combined detected metrics and applied treatments.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

The embodiments described herein are intended to exemplary and non-limiting, the selection of biometric, environmental, or other measured conditions is not limited to a specific metric or multiple metrics described herein but will depend on the particular application and treatment, data collection, and/or other use of the detected metrics. Also, the treatments employed in any of the embodiments described herein is not limited to a specific treatment or action but will depend on the intended use and desired outcome of the combined detected metrics and applied treatments.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

1. A method, comprising:
storing initial baseline biometric data in a memory, where the initial baseline biometric data is obtained using a baseline biometric test;
determining at least one patient-specific threshold for one or more monitored biometric parameters dependent on the stored baseline biometric data;
detecting said one or more monitored biometric parameters using biometric sensors, where the monitored biometric parameters are dependent on at least one physiological change of a patient in response to at least one of a therapeutic treatment and a progression of a disease;
determining at least one exceeded threshold dependent on the detected one or more monitored biometric parameters and the at least one patient-specific threshold; and
activating at least one action depending on the determined exceeded threshold, where the at least one action is at least one of a notification to a patient, a change in the therapeutic treatment, and a notification to at least one trusted receiver.

2. The method of claim 1, where the notification to the trusted receiver includes suggested diagnosis and current treatment options determined through an AI-powered web crawler.

3. The method according to claim 1, wherein the applied treatment includes at least one of an applied electrocuetical treatment for activating a muscle pump of the patient and a pharmaceutical treatment for treating a cardiovascular condition, and wherein the at least one physiological change includes an indication of a change in the cardiovascular condition.

4. The method according to claim 1, wherein the at least one action includes transmitting an alert, modifying the therapeutic treatment, and transmitting data dependent on at least one of the at least one physiological change, the one or more biometric parameters, the therapeutic treatment and the least one patient-specific threshold.

5. The method according to claim 1, wherein the step of determining the at least one patient-specific threshold comprises determining from a data set of the one or more monitored biometric parameters whether the data set is acceptable for deciding that the at least one physiological change threshold has been exceeded.

6. The method according to claim 1, wherein the step of determining the at least one patient-specific threshold further comprises applying a statistical weighting to each of the one or more monitored biometric parameters, where the statistical weighting is dependent on a predetermined value of a ranking of importance in detecting each of the at least one physiological change for said each of the one or more monitored biometric parameters relative to others of the one or more monitored biometric parameters.

7. An apparatus, comprising:
at least one processor; and
at least one memory including computer program code,
the at least one memory and the computer program code configured, with the at least one processor, to cause the apparatus to perform at least the following:
store initial baseline biometric data in a memory, where the initial baseline biometric data is obtained using a baseline biometric test having relatively higher accuracy;
determine at least one patient-specific threshold for one or more monitored biometric parameters dependent on the stored baseline biometric data;
detect said one or more monitored biometric parameters using biometric sensors having relatively lower accuracy than the baseline biometric test, where the biometric parameters are dependent on at least one physiological change of a patient in response to at least one of a therapeutic treatment and a progression of a disease;
determine at least one exceeded threshold dependent on the detected one or more monitored biometric parameters and the at least one patient-specific threshold; and
activate at least one action depending on the determined exceeded threshold, where the at least one action is at least one of a notification to a patient, a change in the therapeutic treatment, and a notification to at least one trusted receiver.

8. The apparatus of claim 7, where the notification to the trusted receiver includes suggested diagnosis and current treatment options determined through an AI-powered web crawler.

9. The apparatus of claim 7, wherein the applied treatment includes at least one of an applied electrocuetical treatment for activating a muscle pump of the patient and an pharmaceutical treatment for treating a cardiovascular condition, and wherein the at least one physiological change includes an indication of a change in the cardiovascular condition.

10. The apparatus of claim 7, wherein the at least one action includes transmitting an alert, modifying the therapeutic treatment, and transmitting data dependent on at least one of the at least one physiological change, the one or more monitored biometric parameters, the therapeutic treatment and the least one patient-specific threshold.

11. The apparatus of claim 7, wherein the step of determining the at least one patient-specific threshold comprises determining from a data set of the one or more monitored biometric parameters whether the data set is acceptable for deciding that the at least one physiological change threshold has been exceeded.

12. The apparatus of claim 7, wherein the step of determining the at least one patient-specific threshold further comprises applying a statistical weighting to each of the one or more monitored biometric parameters, where the statistical weighting is dependent on a predetermined value of a ranking of importance in detecting each of the at least one physiological change for said each of the one or more monitored biometric parameters relative to others of the one or more monitored biometric parameters.

13. A computer program product comprising a computer-readable medium bearing computer program code embodied therein for use with a computer, the computer program code comprising:
code for storing initial baseline biometric data in a memory, where the initial baseline biometric data is obtained using a baseline biometric test having relatively higher accuracy;
determining at least one patient-specific threshold for one or more monitored biometric parameters dependent on the stored baseline biometric data;
detecting said one or more monitored biometric parameters using biometric sensors having relatively lower accuracy than the baseline biometric test, where the biometric parameters are dependent on at least one physiological change of a patient in response to at least one of a therapeutic treatment and a progression of a disease;
determining at least one exceeded threshold dependent on the detected one or more monitored biometric parameters and the at least one patient-specific threshold; and
activating at least one action depending on the determined exceeded threshold, where the at least one action is at least one of a notification to a patient, a change in the therapeutic treatment, and a notification to at least one trusted receiver.

14. The computer program product of claim 13, where the notification to the trusted receiver includes suggested diagnosis and current treatment options determined through an AI-powered web crawler.

15. The computer program product of claim 13, wherein the applied treatment includes at least one of an applied electrocuetical treatment for activating a muscle pump of the patient and an pharmaceutical treatment for treating a cardiovascular condition, and wherein the at least one physiological change includes an indication of a change in the cardiovascular condition.

16. The computer program product of claim 13, wherein the at least one action includes transmitting an alert, modifying the therapeutic treatment, and transmitting data dependent on at least one of the at least one physiological change, the one or more monitored biometric parameters, the therapeutic treatment and the least one patient-specific threshold.

17. The computer program product of claim 13, wherein the step of determining the at least one patient-specific threshold comprises determining from a data set of the one or more monitored biometric parameters whether the data set is acceptable for deciding that the at least one physiological change threshold has been exceeded.

18. The computer program product of claim 13, wherein the step of determining the at least one patient-specific threshold further comprises applying a statistical weighting to each of the one or more monitored biometric parameters, where the statistical weighting is dependent on a predetermined value of a ranking of importance in detecting each of the at least one physiological change for said each of the one or more monitored biometric parameters relative to others of the one or more monitored biometric parameters.

19. The computer program product of claim 13, wherein the therapeutic treatment includes an electrical muscle stimulation signal applied to at least one muscle through the skin surface from at least one electrode in contact with the skin surface, and wherein at least one of the one or more monitored biometric parameters is received from a biometric detector comprising the at least one electrode that applies the electrical muscle stimulation signal.

20. The computer program product of claim 19, wherein the at least one of the one or more monitored biometric parameters is an electronic biometric measurement dependent on heartbeat.

21-50. (canceled)
